(19) United States
(12) Patent Application Publication
(10) Pub. No.: US 2025/0163165 A1
(43) Pub. Date: May 22, 2025

(54) GLYCOSYLATED FORM OF ANTI-IL13R ANTIBODY

(71) Applicant: ASLAN PHARMACEUTICALS PTE LTD, Singapore (SG)

(72) Inventor: Alison WARD, Singapore (SG)

(21) Appl. No.: 18/841,226

(22) PCT Filed: Feb. 23, 2023
(86) PCT No.: PCT/SG2023/050112

§ 371 Date: Aug. 23, 2024

(30) Foreign Application Priority Data
Feb. 23, 2022 (SG) 10202201810Y

(51) Int. Cl. C07K 16/28; C12P 21/00

(52) U.S. Cl. CPC C07K 16/2866; C12P 21/005; C07K 2317/41

(57) ABSTRACT

A glycosylated form of an anti-IL13R antibody or antigen binding fragment thereof and characterized protein populations of any one of the same. Also provided are formulations comprising the glycosylated anti-IL13R antibody or antigen binding fragment thereof, as well as the use of the antibody or antigen binding fragment thereof for treatment.

The present disclosure relates to a glycosylated form of an anti-IL13R antibody or antigen binding fragment thereof and characterized protein populations of any one of the same. Also provided are formulations comprising the antibody or antigen binding fragment thereof, as well as the use of the antibody or antigen binding fragment thereof for treatment.

### BACKGROUND

The biological mechanisms of the body are very finely balanced and signalling is an intricate network of receptors and ligands. Carbohydrates, for example on proteins are involved in sophisticated signalling mechanisms, for example they are involved in recruiting effector function in the Fc region of antibodies. Glycans are also known to have a major influence on protein antigen uptake, proteolytic processing and immune responses.

Glycosylation also has an influence on the folding, stability and/or half-life of the antibody.

The review paper “Glycans as a Key Checkpoints of T Cell Activity and Function” Frontiers in Immunology November 2018, Vol 9 Article 2754 states:

The immune system is highly controlled and fine-tuned by glycosylation, through the addition of a diversity of carbohydrates structures (glycans) to virtually all immune cell receptors. Despite a relative backlog in understanding the importance of glycans in the immune system, due to its inherent complexity, remarkable findings have been highlighting the essential contributions of glycosylation in the regulation of both innate and adaptive immune responses with important implications in the pathogenesis of major diseases such as autoimmunity and cancer. Glycans are implicated in fundamental cellular and molecular processes that regulate both stimulatory and inhibitory immune pathways. Besides being actively involved in pathogen recognition through interaction with glycan-binding proteins (such as C-type lectins), glycans have been also shown to regulate key pathophysiological steps within T cell biology such as T cell development and thymocyte selection; T cell activity and signalling as well as T cell differentiation and proliferation. These effects of glycans in T cells functions highlight their importance as determinants of either self-tolerance or T cell hyper-responsiveness which ultimately might be implicated in the creation of tolerogenic pathways in cancer or loss of immunological tolerance in autoimmunity. This review discusses how specific glycans (with a focus on N-linked glycans) act as regulators of T cell biology and their implications in disease.

The signals to the immune system from glycans can be agonistic or antagonistic.

Eblasakimab (previously known as ASLAN004 and described in WO2008/060813 as antibody 10G5-6) is an anti-IL13R antibody which has been shown to effectively antagonise IL-13 function through inhibiting the binding of IL-13 to its receptor IL-13Rα1 and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

Eblasakimab is thought to be useful in the treatment of atopic dermatitis, an allergic/autoimmune type indication. As explained above, given the crucial role of N-glycosylation in the function in pharmacokinetics of antibodies and signalling/regulation of immune responses, the present inventors believe that it is vitally important, for example for protein folding, stability, immunogenicity, off-target effects and/or therapeutic activity of eblasakimab, to closely define the desired glycosylation pattern.

### SUMMARY OF DISCLOSURE

The present invention is summarized in the following paragraphs:
1. An anti-IL-13R antibody or antigen binding fragment thereof, comprising:
   a. a VH CDR1 sequence as set forth in SEQ ID NO: 1, a VH CDR2; sequence as set forth in SEQ ID NO: 2, a VH CDR3 s set forth in SEQ ID NO: 10,
   b. a VL CDR1 as set forth in SEQ ID NO: 31, a VL CDR2 sequence as set forth in SEQ ID NO: 32, and a VL CDR3 set forth in SEQ ID NO: 45;
   c. a heavy chain constant region domain comprising the sequence EEQFNSTYR SEQ ID NO: 65
   wherein the N in SEQ ID NO: 64 is linked to a glycan (i.e. an N-glycan).
2. The antibody or antigen binding fragment thereof according to paragraph 1, wherein the glycan comprises in the range 5 to 11 saccharides (sugar molecules), for example 5, 6, 7, 8, 9, 10 or 11, such as 6, 7, 8, 9 or 10, in particular 7 or 8.
3. The antibody or antigen binding fragment thereof according to paragraph 1 or 2, wherein the glycan comprises 1 to 4 N-acetylglucosamine(s) (GlcAc), for example 1 to 4 N-acetylglucosamine, such as 1, 2, 3 or 4 N-acetylglucosamines, in particular 2 or 4.
4. The antibody or antigen binding fragment according to paragraph 3, wherein an N-acetylglucosamine is bonding to the N in SEQ ID NO: 64.
5. The antibody or antigen binding fragment thereof according to any one of paragraphs 1 to 4, wherein the glycan comprises mannose, for example 1 to 5 mannose, such as 1, 2, 3, 4 or 5 mannose, more specifically 3 or 5 mannose, in particular 3 mannoses.
6. The antibody or antigen binding fragment thereof according to any one of paragraphs 1 to 5, where the glycan further comprises galactose, for example 1 to 3 galactose, such as 1 or 2.
7. The antibody or antigen binding fragment thereof according to paragraph 6, wherein galactose is the terminating saccharide free (unlinked) end of the glycan.
8. The antibody or binding fragment according to any one of paragraphs 1 to 5, wherein the glycan does not comprise galactose.
9. The antibody or antigen binding fragment thereof according to any one of paragraphs 1 to 8, wherein the glycan comprises fucose, for example 1 to 2 fucose, such as 1.
10. The antibody or antigen binding fragment thereof according to paragraph 9, wherein a fucose is linked to an N-acetylglucosamine.
11. The antibody or antigen binding fragment according to paragraph 10, wherein the N-acetylglucosamine is bonding the N in SEQ ID NO: 64.
12. The antibody or antigen binding fragment thereof according to any one of paragraphs 1 to 8, wherein the N-glycan does not comprise fucose.
13. The antibody or antigen binding fragment thereof according to any one of paragraphs 1 to 12, wherein the glycan is selected from the group comprising: G0-N, G0F-N, G0, G0F, M5, G1F, G1F′ G2F.
14. The antibody or antigen binding fragment thereof according to any one of paragraphs 1 to 13, wherein the N-glycan is selected from the group comprising G0F-N, G0, G0F, M5, G1F and G1F′.
15. The antibody or antigen binding fragment thereof according to any one of paragraphs 1 to 14, wherein the N-glycan is selected from the group comprising G0F-N, G0F, G1F and G1F′.
16. The antibody or antigen binding fragment thereof according to any one of paragraphs 1 to 15, wherein the N-glycan is G0F.
17. The antibody or antigen binding fragment thereof according to any one of paragraphs 1 to 16, wherein the anti-IL13R antibody or antigen binding fragment thereof comprises a VH domain with a sequence shown in SEQ ID NO: 51 or a sequence at least 95% identical thereto.
18. The antibody or antigen binding fragment thereof according to any one of paragraphs 1 to 17, wherein the anti-IL13R antibody or antigen binding fragment thereof comprises a VL domain with a sequence shown in SEQ ID NO: 53 or a sequence at least 95% identical thereto.
19. A composition of antibodies or antigen binding fragments thereof as defined in any one of paragraphs 1 to 18, wherein the composition is characterized by a population of glycans, such that G0F comprises at least 50% of said population, for example 60, 65, 70, 75, 80, 81, 82, 83, 84, 85, 86, 87, 88, 89 or 90% of the population, in particular 80 to 90%, such as 81, 82, 83, 84, 85 or 86%.
20. A composition according to paragraph 19, wherein the population of glycans comprises G0F-N, for example 1 to 5% of the population, such as 2 to 4%, for example 2.5 to 3.9%, in particular 2.5, 3.0, 3.7 or 3.8%, such as 3.7%.
21. A composition according to paragraph 19 or 20, wherein the population of glycans comprises G1F′, for example 1 to 5% of the population, such as 3.5 to 3.9%, in particular 3.7%.
22. A composition according to any one of paragraphs 19 to 21, wherein the population of glycans comprises G1F, for example 1 to 5% of the population, such as 3.2 to 3.2%, in particular 3.5%.
23. A composition according to any one of paragraphs 19 to 22, wherein the population of glycans comprises G0, for example 1 to 3% of the population, such as 1.7 to 2.1%, in particular 1.9%.
24. A composition according to any one of paragraphs 19 to 23, wherein the population of glycans comprises M5, for example 0.5 to 1.5% of the population, such as 0.9 to 1.3%, in particular 0.9, 1.0, 1.1 or 1.3, such as 1.1%.
25. A composition according to any one of paragraphs 19 to 24, wherein the population of glycans comprises G0-N, for example 0.2 to 1.2% of the population, such as 0.5 to 0.9%, in particular 0.7%.
26. A composition according to any one of paragraphs 19 to 25, wherein the population of glycans comprises G2F, for example 0.1 to 1% of the population, such as 0.3 to 0.7, in particular 0.5%.
27. A composition according to any one of paragraphs 19 to 26, wherein the population of glycans comprises afucosylated glycans (such as G0-N and/or G0), for example 1.0 to 3.0% of the population, such as 1.5 to 2.6%, such as 1.8, 2.0 or 2.6%, in particular 2.6%.
28. A composition according to any one of paragraphs 19 to 27, wherein the population of glycans comprises galactosylated glycans (such as G1F, G1F′ and/or G2F), for example 6 to 10% of the population, such as 7 to 9%, such as 7.3, 7.7, 8.1 or 8.8%, in particular 7.7%.
29. A composition according to any one of claims 19 to 28, which further comprise a pharmaceutically acceptable excipient, diluent or carrier.
30. A pharmaceutical formulation comprising one or more antibodies or antigen binding fragments thereof according to any one of paragraphs 1 to 18 and a pharmaceutically acceptable excipient, diluent or carrier.
31. A method of treating an inflammatory disorder comprising administering a therapeutically effective amount of an antibody or antigen binding fragment thereof or composition according to any one of paragraphs 1 to 28 or a formulation according to paragraph 30 to a subject in need thereof.
32. The method according to paragraph 31, for use in the treatment of inflammatory disease, for example wherein the inflammatory disorder is atopic dermatitis, such as moderate to severe atopic dermatitis.
33. An antibody or antigen binding fragment thereof according to any one of paragraphs 1 to 18, a composition according to any one of paragraphs 19 to 29 or a formulation according to paragraph 30, for use in treatment, for example use in the treatment of inflammatory disease, in particular the treatment of atopic dermatitis, such as moderate to severe atopic dermatitis.
34. Use of an antibody or antigen binding fragment thereof according to any one of paragraphs 1 to 18, a composition according to any one of paragraphs 19 to 29 or a formulation according to paragraph 30, in the manufacture of a medicament for the treatment of inflammatory disease, for example atopic dermatitis, such as moderate to severe atopic dermatitis.

### DETAILED DESCRIPTION

One of the factors influencing the glycosylation pattern of the antibody is the choice of manufacturing cell line. A number of cells lines can be employed. Examples of such cell lines Include eukaryotic cell lines, such as mammalian cells lines. In one embodiment, the manufacturing cell line is a mammalian cell line, for example selected from the group comprising Chinese Hamster Cell (CHO), human embryonic kidney (HEK), such as HEK293, mouse myeloma Sp2/0, human HT-1080, human lymphoblastoid, and NSO.

Chinese Hamster Cell (CHO) lines in particular produce a desirable glycosylation pattern when employed to recombinantly express the antibody and antigen binding fragments of the present disclosure. Thus, in one embodiment, the antibody or antigen binding fragments of the present disclosure are produced using CHO cell lines.

Saccharide (also referred to as sugar molecules) as employed herein refers to a single sugar monomer, for example fructose, galactose, mannose, N-acetylglucosamine, N-acetylgalactosamine, sialic acid, xylose, glucuronic acid, iduronic acid, N-acetylneuraminic acid, N-glycolyl neuraminic acid.

Terminating saccharide at the free end of the glycan as employed herein refers to the free end of the carbohydrate that is not linked to the macromolecule. It will be clear to the skilled person that where the glycan is branched there will be terminating saccharide at the end of each branch, which together are considered the “free end” unless the context indicates otherwise.

### Glycosylation

Glycosylation is the process by which a carbohydrate (an oligosaccharide or polysaccharide) is covalently attached to a target macromolecule, such as a protein or a lipid. The appended carbohydrate is referred to as a glycan.

The glycan can be linked to through an oxygen or nitrogen in the macromolecule.

These modifications serve various functions. For example some proteins do not fold correctly or are unstable unless they are glycosylated. The influence of glycosylation on the folding and stability of glycoprotein is twofold. Firstly, the highly soluble glycans may have a direct physicochemical stabilisation effect. Secondly, N-linked glycans mediate a critical quality control check point in glycoprotein folding in the endoplasmic reticulum.

Glycosylation also plays a role in cell-to-cell recognition and adhesion via sugar-binding proteins called lectins, which recognize specific carbohydrate moieties. Thus, glycosylation is an important parameter in the optimization of many glycoprotein-based drugs such as monoclonal antibodies which function by binding to target antigens.

Glycosylation also underpins the ABO blood group system. It is the presence or absence of glycosyltransferases which dictates which blood group antigens are presented and hence what antibody specificities are exhibited. This immunological role may well have driven the diversification of glycan heterogeneity and creates a barrier to zoonotic transmission of viruses. In addition, glycosylation is often used by viruses to shield the underlying viral protein from immune recognition.

Glycosylation may also modulate the thermodynamic and kinetic stability of proteins.

Glycosylation types are classified according to the identity of the atom of the amino acid which binds the carbohydrate, i.e., C-linked glycosylation, N-linked glycosylation, O-linked glycosylation or S-linked glycosylation. N-, C- and S-glycosylation take place in the endoplasmic reticulum and/or the Golgi apparatus and only extracellular or secreted proteins are concerned. In contrast, both intracellular and extracellular proteins can be O-glycosylated. The present disclosure is primarily concerned with N-linked glycosylation.

### N-Linked Glycosylation

Eukaryotes commonly attach glycans in the endoplasmic reticulum to the nitrogen (N) in the side chain of a protein asparagine residue by a β-1N linkage. The asparagine residue typically occurs in the sequence Asn-Xaa-Ser/Thr/Cys (where Xaa represents any amino acid), in single letter amino acid code: NXS/T, where X is any amino acid. The process of attaching the glycan to the nitrogen in the asparagine residue is known as ‘N-linked glycosylation’ while the glycans attached are known as ‘N-glycans’.

N-glycosylation occurs mainly in eukaryotes and in archaea—most bacteria are unable to perform this type of glycosylation.

In one embodiment the anti-IL13R antibody or antigen binding fragment thereof comprises a heavy chain constant region domain comprising the sequence EEQFNSTYR (SEQ ID NO: 64), wherein the N is linked to a N-glycan.

### N-Glycans

N-glycans are based on the common core pentasaccharide, Man₃GlcNAc₂. Following attachment of the N-glycan in the endoplasmic reticulum, further modifications may occur in the golgi. Typically, these modifications occur via an ordered sequence of enzymatic reactions, known as a cascade. Different organisms provide different glycosylation enzymes (glycosyltransferases and glycosidases) and different glycosyl substrates, so that the final composition of a sugar side chain may vary markedly depending upon the host. These modifications result in 3 main classes of N-glycans: High-mannose, Hybrid and Complex:

Microorganisms such as filamentous fungi and yeast (lower eukaryotes) typically add additional mannose and/or mannosylphosphate sugars. The resulting glycan is known as a “high-mannose” N-glycans.

Hybrid N-glycans are characterised as containing both unsubstituted terminal mannose residues (such as those present in high-mannose N-glycans), and substituted mannose residues with a N-acetylglucosamine linkage (such as those present in complex N-glycans).

Complex N-glycans are primarily produced in humans and animals. A complex N-glycan refers to a structure with typically two to six outer branches with a sialyllactosamine sequence linked to the inner Man₃GloNAc₂ core structure. A complex N-glycan has at least one branch, and preferably at least two, of alternating N-Acetylglucosamine (GlcNAc) and galactose (Gal) residues that terminate in oligosaccharides such as, for example: NeuNAc-; NeuAcα:2-6GalNAcα1-; NeuAcα2-3Galβ1-3GalNAcα1-; NeuAcα2-3/6Galβ1-4GlcNAcβ1-; GlcNAcα1-4Galβ1-(mucins only); Fucα1-2Galβ1-(blood group H). Sulfate esters can occur on galactose, GalNAc, and GlcNAc residues, and phosphate esters can occur on mannose residues. NeuAc (Neu: neuraminic acid; Ac: acetyl) can be O-acetylated or replaced by NeuGI (N-glycolylneuraminic acid). Complex N-glycans may also have intrachain substitutions of bisecting GlcNAc and core fucose (Fac).

Thus, in one embodiment, the N-glycan is a complex N-glycan.

In one embodiment the N-glycan comprises 2 to 4 GlcNac.

In one embodiment the N-glycan comprises mannose, for example 3-5 mannose.

In one embodiment the N-glycan further comprises galactose, for example 1 or 2, i.e. the N-glycan is galactosylated.

As used herein, the term galactosylation refers to the addition of galactose to a N-glycan structure. Examples of such N-glycans include but are not limited to G1F, G1F′ and G2F. Thus, in one embodiment, the antibody or antigen binding fragment of the present disclosure is galactosylated. Thus, in one embodiment the antibody or antigen binding fragment of the present disclosure comprises N-glycan galactosylation. In one embodiment the N-glycan is a galactosylated N-glycan, such as G1F, G1F′ and/or G2F. Hence in one embodiment the N-glycan is selected from the group comprising G1F, G1F′ and G2F.

In one embodiment the N-glycan comprises fucose. In one embodiment the N-glycan does not comprise fucose, i.e. the N-glycan is afucosylated.

As used herein, the term afucosylation refers to the lack of fucose sugar units in a N-glycan. Examples of such N-glycans include but are not limited to G0-N and G0. Thus, in one embodiment, the antibody or antigen binding fragment of the present disclosure is afucosylated. Thus, in one embodiment the antibody or antigen binding fragment of the present disclosure comprises N-glycan afucosylation. In one embodiment the N-glycan is an afucosylated N-glycan, such as G0-N and/or G0. Hence, in one embodiment the N-glycan is selected from the group comprising G0-N and G0.

To make naming and representing N-glycans (in particular complex N-glycans) easier, scientists have developed two shorthand notations known as the Essentials and Oxford nomenclature systems:

The Essentials System is named after “Essentials of Glycobiology,” the textbook in which it was first published in 1978. The notation represents individual sugars as coloured symbols and depicts how they connect in the glycan. Over the years, scientists have expanded it to include additional symbols for most common monosaccharides found in nature. Scientists sometimes include linkage information along the glycosidic bonds connecting the sugar symbols. They use “a” and “B” to represent the two stereochemical types of glycosidic bonds and numbers to denote the ring position of the carbon on the sugar on which the glycosidic bond originates. But linkage information can be omitted in the notation.

The Oxford System was designed by scientists at the University of Oxford's Glycobiology Institute in 2009 to be readable in black and white. Some of the Oxford scheme's monosaccharide symbols differ from those in the Essentials scheme. Linkages between sugars are encoded as dashed lines for a stereochemistry or solid lines for B. The angles of the lines denote the ring position where the bond originates. The newly standardized Essentials system permits an optional mixed form of notation in which the Oxford system's angles and dashed and solid bonds are used but its sugar symbols are not.

A detailed discussion on N-glycan nomenclature available at http://www.imgt.org/IMGTeducation/IMGTlexique/G/Glycosylation.html. The Essentials System is the more widely used of the two and is used within the context of the present disclosure.

In one embodiment, the N-glycan is selected from the group comprising: G0-N, G0F-N, G0, G0F, M5, G1F, G1F′ and G2F. In one embodiment, the N-glycan is selected from the group comprising G0F-N, G0F, G1F and G1F′. In one embodiment, the N-glycan is G0F.

As used herein, the term “G0-N” used interchangeably with “A1” refers to the oligosaccharide structure:

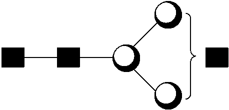

As used herein, the term “G0F-N” used interchangeable with “F(6)A1” refers to the oligosaccharide structure:

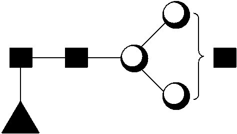

As used herein, the term “G0” used interchangeably with “A2” refers to the oligosaccharide structure

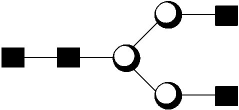

As used herein, the term “G0F” used interchangeably with “F(6)A2” refers to the oligosaccharide structure:

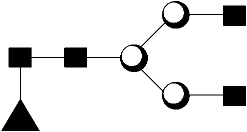

As used herein, the term “M5” used interchangeably with “Man5” refers to the oligosaccharide structure:

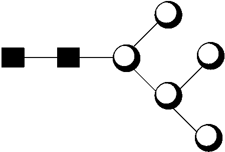

As used herein, the term “G1F” used interchangeably with “FA2(6)G1” refers to the oligosaccharide structure:

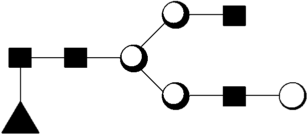

As used herein, the term “G1F” used interchangeably with “FA2(3)G1” refers to the oligosaccharide structure:

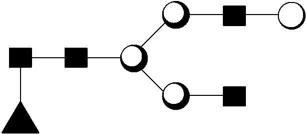

As used herein, the term “G2F” used interchangeably with “F(6)A2G(4)2” refers to the oligosaccharide structure:

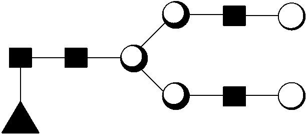

### Therapeutic Glycoproteins

A significant fraction of proteins isolated from humans or other animals are glycosylated. Among proteins used therapeutically, about 70% are glycosylated. If a therapeutic protein is produced in a microorganism host such as yeast, however, and is glycosylated utilizing the endogenous pathway, its therapeutic efficiency is typically greatly reduced. Such glycoproteins are typically immunogenic in humans and show a reduced half-life in vivo after administration (Takeuchi, 1997).

Specific receptors in humans and animals can recognize terminal mannose residues and promote the rapid clearance of the protein from the bloodstream. Additional adverse effects may include changes in protein folding, solubility, susceptibility to proteases, trafficking, transport, compartmentalization, secretion, recognition by other proteins or factors, antigenicity, or allergenicity. Accordingly, it has been necessary to produce therapeutic glycoproteins in animal host systems, so that the pattern of glycosylation is identical or at least similar to that in humans or in the intended recipient species. In most cases a mammalian host system, such as mammalian cell culture, is used. In the context of the present disclosure, eblasakimab is a therapeutic glycoprotein.

### Systems for Producing Therapeutic Glycoproteins

In order to produce therapeutic proteins that have appropriate glycoforms and have satisfactory therapeutic effects, animal or plant-based expression systems have been used. Examples of suitable systems include but are not limited to:
1. Chinese hamster ovary cells (CHO), mouse fibroblast cells and mouse myeloma cells (Arzneimittelforschung, 1998 August; 48 (8): 870-880);
2. transgenic animals such as goats, sheep, mice and others (Dente Prog. Clin. Biol. 1989 Res. 300:85-98, Ruther et al., 1988 Cell 53 (6): 847-856; Ware, J., et al. 1993 Thrombosis and Haemostasis 69 (6): 1194-1194; Cole, E. S., et al. 1994 J. Cell. Biochem. 265-265);
3. plants (Arabidopsis thaliana, tobacco etc.) (Staub, et al. 2000 Nature Biotechnology 18 (3): 333-338) (McGarvey, P. B., et al. 1995 Bio-Technology 13 (13): 1484-1487; Bardor, M., et al. 1999 Trends in Plant Science 4 (9): 376-380);
4. insect cells (Spodoptera frugiperda Sf9, S121, Trichoplusia ni, etc. in combination with recombinant baculoviruses such as Autographa californica multiple nuclear polyhedrosis virus which infects lepidopteran cells) (Altmans et al., 1999 Glycoconj. J. 16 (2): 109-123).

Recombinant human proteins expressed in the above-mentioned host systems may still include non-human glycoforms (Raju et al., 2000 Annals Biochem. 283 (2): 123-132), In particular, fraction of the N-glycans may lack terminal sialic acid, typically found in human glycoproteins. Substantial efforts have been directed to developing processes to obtain glycoproteins that are as close as possible in structure to the human forms, or have other therapeutic advantages. Glycoproteins having specific glycoforms may be especially useful, for example in the targeting of therapeutic proteins. For example, the addition of one or more sialic acid residues to a glycan side chain may increase the lifetime of a therapeutic glycoprotein in vivo after administration.

Accordingly, the mammalian host cells may be genetically engineered to increase the extent of terminal sialic acid in glycoproteins expressed in the cells. Alternatively sialic acid may be conjugated to the protein of interest in vitro prior to administration using a sialic acid transferase and an appropriate substrate. In addition, changes in growth medium composition or the expression of enzymes involved in human glycosylation have been employed to produce glycoproteins more closely resembling the human forms (S. Weikert, et al., Nature Biotechnology, 1999, 17, 1116-1121; Werner, Noe, et al 1998 Arzneimittelforschung 48 (8): 870-880; Weikert, Papac et al., 1999; Andersen and Goochee 1994 Cur, Opin. Biotechnol. 5:546-549; Yang and Butler 2000 Biotechnol. Bioengin. 68 (4): 370-380). Alternatively cultured human cells may be used.

The skilled addressee is also aware of other suitable expression systems for the production of therapeutic glycoproteins not explicitly mentioned above.

In one embodiment, the antibody or antigen binding fragment of the present disclosure is produced using the process described in Example 1.

In one embodiment, the antibody or antigen binding fragment of the present disclosure is produced using the manufacturing process as outlined in FIG. 1.

In one embodiment, the antibody or antigen binding fragments of the present disclosure are produced using CHO cell lines.

In one embodiment the cells are grown in a selective medium is as described in Example 1, such as during the thawing stage, seed train stage and/or expansion stage. Thus, in one embodiment the selective medium comprises BalanCD CHO Growth A, L-glutamine, sodium bicarbonate and an antibiotic, such as puromycin and/or hygromycin. In one embodiment, the selective medium has pH of about 6.8 to about 7.2, such as 6.8, 6.9, 7.0, 7.1 or 7.2.

In one embodiment the cells are grown in a production medium is as described in Example 1, such as during the production stage. Thus, in one embodiment the production medium comprises BalanCD CHO Growth A, L-glutamine and sodium bicarbonate. In one embodiment, the production medium has pH of about 6.8 to about 7.2, such as 6.8, 6.9, 7.0, 7.1 or 7.2.

In one embodiment the initial cell density at the thawing stage is about 4±1×10⁵ cells/ml, such as 3×10⁵, 3.5×10⁵, 4.0×10⁵, 4.5×10⁵, 5.0×10⁵ cells/ml. In one embodiment the inoculated cell density for each cell passage is 5±1×10⁵ cells/ml, such as 4.0×10⁵, 4.5×10⁵, 5.0×10⁵, 5.5×10⁵, 6.0×10⁵ cells/ml.

In one embodiment the cells at the thawing stage, seed train stage and/or expansion stage are cultivated until cell densities are 40×10⁵ to 100×10⁵ cells/ml, such as 40×10⁵, 50×10⁵, 60×10⁵, 70×10⁵, 80×10⁵, 90×10⁵, or 100×10⁵ cells/ml. In one embodiment the cells' viability is ≥90%, such as 90%, 91%, 92%, 93%, 94%, 95%, 96%, 97%, 98%, 99% or 100%.

In one embodiment the cells are cultivated for 2 to 4 days during the thawing and/or seed train stages. In one embodiment the cells are cultivated for 3 to 5 days during the expansion stage.

In one embodiment the cells are maintained at a viable cell density (VCD) such that the viability of the cells is ≥90%.

In one embodiment the cells are grown at pH 7.00±0.20, for example 6.8, 6.9, 7.0, 7.1 or 7.2 during the expansion stage. In one embodiment the cells are grown at pH 7.00±0.05, for example 6.95, 6.96, 6.97, 6.98, 6.99, 7.00, 7.01, 7.02, 7.03, 7.04 or 7.05 during the production stage.

In one embodiment the pH is controlled by adding sodium carbonate, such as 1M sodium carbonate solution, lactic acid, such as 20% (v/v) lactic acid or a combination of both to the culture medium.

In one embodiment the cells are grown at about 37° C., such as 36, 36.5, 37 or 37.5° C., in particular at 37° C. In one embodiment the cells are grown at about 5% (v/v), such as 4.5, 4.6, 4.7, 4.8, 4.9, 5.0, 5.1, 5.2, 5.3, 5.4 or 5.5%, in particular at 5% CO₂ concentration.

In one embodiment the cells are grown at 37° C. such as 36, 36.5, 37 or 37.5° C., in particular at 37° C. from Day 0 to Day 5 during the production stage. In one embodiment the cells are grown at 33° C., such as 32, 32.5, 33 or 33.5° C. from Day 6 to Day 12 of the production stage.

In one embodiment the cells are fed with one or more of Cell Boost 7a, Cell Boost 7b and glucose, such as 30% (v/v) glucose solution during the production stage. The composition of Cell Boost 7a and Cell Boost 7b is described in Example 1. In one embodiment Cell Boost 7a has a pH of 6.7±0.1, such as 6.6, 6.7 or 6.8. In one embodiment Cell Boost 7b has a pH of 11.0 to 11.4, such as 11, 11.1, 11.2, 11.3 or 11.4.

In one embodiment the feeding strategy is as described in Example 1. Thus, in one embodiment the glucose is maintained at a concentration of about 4 g/L, such as 3.8, 3.9, 4.0, 4.1 or 4.2 g/L. In one embodiment the Cell Boost 7a comprises about 3%, such as 2.5, 2.6, 2.7, 2.8, 2.9, 3.0, 3.1, 3.2, 3.3, 3.4 or 3.5% of the initial working volume, for example from Day 3 to Day 11. In one embodiment the Cell Boost 7b comprises about 0.3%, such as 0.25, 0.26, 0.27, 0.28, 0.29, 0.30, 0.31, 0.32, 0.33, 0.34 or 0.35% of the initial working volume, for example from Day 3 to Day 11.

In one embodiment the cells are cultured for about 12 days before they are harvested, for example 11, 12 or 13 days.

### Anti-IL13R Antibody

Interleukin-13 receptor (IL-13R) as used herein is a type I cytokine receptor, which binds to Interleukin-13. It consists of two subunits, encoded by IL13Rα1 and IL4R, respectively. These two genes encode the proteins IL-13Rα1 and IL-4Rα. These form a dimer (also known as the type II receptor complex) with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STAT6. Human IL-13Rα1 has the Uniprot number P3597.

IL-13Rα2, previously called IL-13R and IL-13Rα, is another receptor which is able to bind to IL-13. However, in contrast to IL-13Rα1, this protein binds IL-13 with high affinity, but it does not bind IL-4. Human IL-13Rα2 has the Uniprot number Q14627.

Anti-IL13R antibody as used herein refers to an antibody that has specificity for IL13R, for example IL13Rα1 or IL13Rα2.

In one embodiment, the anti-IL13R antibody of the present disclosure is specific for IL13Rα1. Thus, in one embodiment the presently disclosed antibody or antigen binding fragment thereof is an anti-IL13Rα1 antibody or binding fragment thereof. In one embodiment, the anti-IL13R antibody binds to an epitope comprising the amino acid sequence FFYQ.

The anti-IL13R antibodies of the present disclosure may comprise a complete antibody molecule having full length heavy and light chains or a binding fragment thereof. Binding fragments include but are not limited to Fab, modified Fab, Fab′, F(ab′)₂, Fv, single domain antibodies (such as VH, VL, VHH, IgNAR V domains), scFv, bi, tri or tetra-valent antibodies, Bis-scFv, diabodies, triabodies, tetrabodies and epitope-binding fragments of any of the above (see for example Holliger and Hudson, 2005, Nature Biotech. 23 (9): 1126-1136; Adair and Lawson, 2005, Drug Design Reviews—Online 2 (3), 209-217).

The methods for creating and manufacturing these antibody fragments are well known in the art (see for example Verma et al, 1998, Journal of Immunological Methods, 216, 165-181). Other antibody fragments for use in the present invention include the Fab and Fab′ fragments described in WO2005/003169, WO2005/003170 and WO2005/003171. Other antibody fragments for use in the present invention include Fab-Fv and Fab-dsFv fragments described in WO2010/035012 and antibody fragments comprising those fragments. Multi-valent antibodies may comprise multiple specificities or may be monospecific (see for example WO 92/22853 and WO05/113605).

The antibody and fragments thereof, for use in the present disclosure may be from any species including for example mouse, rat, shark, rabbit, pig, hamster, camel, llama, goat or human. Chimeric antibodies have a non-human variable regions and human constant regions.

An antibody or binding fragment for use in the present invention can be derived from any class (e.g. IgG, IgE, IgM, IgD or IgA) or subclass of immunoglobulin molecule. In one embodiment the antibody employed in the present disclosure is IgG4 or IgG4 with a 241P mutation.

In one embodiment the antibody or binding fragment employed in the formulation of the present disclosure has affinity of 5 nM or higher (higher affinity is a lower numerical value), for example 500 pM, such as 250 pM or higher, in particular 125 pM or less.

In one embodiment CDRH1 is an amino acid sequence GYSFTSYWIG (SEQ ID NO: 1).

In one embodiment CDRH2 is an amino acid sequence VIYPGDSYTR (SEQ ID NO: 2)

In one embodiment CDRH3 has the formula:

X₁ Pro Asn Trp Gly X₆ X₇ Asp X₉ SEQ ID NO: 3
X₁ denotes Phe, Met, Gln, Leu or Val
X₆ denotes Ser or Ala
X₇ denotes Phe, Leu, Ala or Met
X₉ denotes Tyr, Gln, Lys, Arg, Trp, His, Ala,
Thr, Ser, Asn or Gly

In one embodiment the IL13-R1α1 antibody or binding fragment employed in the formulation of the present disclosure comprises a CDRH3 in dependently selected from SEQ ID NO: 4 to 30. MPNWGSLDH (SEQ ID NO: 10)

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 3. In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 30. In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

In one embodiment CDRL1 is an amino acid sequence RASQSISSSYLA (SEQ ID NO: 31).

In one embodiment CDRL2 is an amino acid sequence GASSRAT (SEQ ID NO: 32).

In one embodiment CDL3 has the formula:

Gln X₂X₃X₄X₅ SEQ ID NO: 33
X₂ denotes Gln, Arg, Met, Ser, Thr or Val.
X₃ denotes Tyr or Val.
X₄ denotes Glu, Ala, Gly or Ser.
X₅ denotes Thr, Ala or Ser.

In one embodiment the IL-13Rα1 antibody employed in the formulation of the present disclosure comprises a CDRL3 in dependently selected from SEQ ID NO: 34 to 47. QQYAS (SEQ ID NO: 45)

In one embodiment, the anti-IL-13Rα antibody or binding fragment employed in the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33. In one embodiment, the anti-IL-13Rα antibody of the present disclosure comprises a VL CDR1 comprising an amino acid sequence SEQ ID NO: 84, a VL CDR2 comprising an amino acid sequence SEQ ID NO: 85, and a VL CDR3 comprising an amino acid sequence as set forth in SEQ ID NO: 34 35, 36, 37, 38, 39, 40, 41, 42, 43, 44, 45, 46, or 47. In one embodiment, the anti-IL-13Rα antibody of the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 47. In one embodiment, the anti-IL-13Rα antibody of the present disclosure comprises a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45. In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: or 3, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33. In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 30, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 34, 35, 36, 37, 38, 39, 40, 41, 42, 43, 44, 45, 46, or 47. In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 30, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 47. In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 30, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 47.

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment the VH region is independently selected from a sequence from the group comprising: SEQ ID NO: 48; SEQ ID NO: 49; SEQ ID NO: 50; SEQ ID NO: 51 and a sequence at least 95% identical to any one of the same.

SEQ ID NO: 51:
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSS

In one embodiment the VL is independently selected from a sequence from the group comprising: SEQ ID NO: 52, SEQ ID NO: 53, SEQ ID NO: 54 and a sequence at least 95% identical to any one of the same (*K can be deleted in a post translational modification for example from the C-terminus)

SEQ ID NO: 53
EIVLTQSPGTLSLSPGERATLSCRASQSISSSYLAWYQQKPGQAPRLLI
YGASSRATGIPDRFSGSGSGTDFTLTISRLEPEDFAVYYCQQYASFGQG
TKVEIK

In one embodiment the VH sequence is SEQ ID NO: 48 (or a sequence at least 95% identical thereto) and the VL sequence is SEQ ID NO: 52, SEQ ID NO: 53, or SEQ ID NO: 54 (or a sequence at least 95% identical to any one of the same). In one embodiment the VH sequence is SEQ ID NO: 49 (or a sequence at least 95% identical thereto) and the VL sequence is SEQ ID NO: 52, SEQ ID NO: 53, or SEQ ID NO: 54 (or a sequence at least 95% identical to any one of the same). In one embodiment the VH sequence is SEQ ID NO: 50 (or a sequence at least 95% identical thereto) and the VL sequence is SEQ ID NO: 52, SEQ ID NO: 53, or SEQ ID NO: 54 (or a sequence at least 95% identical to any one of the same). In one embodiment the VH sequence is SEQ ID NO: 51 (or a sequence at least 95% identical thereto) and the VL sequence is SEQ ID NO: 52, SEQ ID NO: 53, or SEQ ID NO: 54 (or a sequence at least 95% identical to any one of the same). In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same). In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same). In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same). In one embodiment the VH sequence is SEQ ID NO: 51 (or a sequence at least 95% identical thereto) and the VL sequence is SEQ ID NO: 53 (or a sequence at least 95% identical thereto).

Variable region as employed herein refers to the region in an antibody chain comprising the CDRs and a suitable framework.

In one embodiment the heavy chain comprises a sequence independently selected from the group comprising: SEQ ID NO: 55, SEQ ID NO: 56, SEQ ID NO: 57, SEQ ID NO: 58, SEQ ID NO: 59, SEQ ID NO: 60, and a sequence at least 95% identical to any one of the same (K can deleted in a post translational modification for example from the C-terminus).

In one embodiment the light chain is independently selected from a group comprising: SEQ ID NO: 61: SEQ ID NO: 62, SEQ ID NO: 63 and a sequence at least 95% identical to any one of the same.

In one embodiment the heavy chain is independently selected from SEQ ID NO: 55, 56, 57, 58, 59 and 60 (or a sequence at least 95% identical to any one of the same) and the light chain is independently selected from SEQ ID NO: 61, 62 and 63 (or a sequence at least 95% identical to any one of the same). In one embodiment the heavy chain is SEQ ID NO: 55 (or a sequence at least 95% identical thereto) and the light chain is independently selected from SEQ ID NO: 61, 62 and 63 (or a sequence at least 95% identical to any one of the same). In one embodiment the heavy chain is SEQ ID NO: 56 (or a sequence at least 95% identical thereto) and the light chain is independently selected from SEQ ID NO: 61, 62 and 63 (or a sequence at least 95% identical to any one of the same). In one embodiment the heavy chain is SEQ ID NO: 57 (or a sequence at least 95% identical thereto) and the light chain is independently selected from SEQ ID NO: 61, 62 and 63 (or a sequence at least 95% identical to any one of the same). In one embodiment the heavy chain is SEQ ID NO: 58 (or a sequence at least 95% identical thereto) and the light chain is independently selected from SEQ ID NO: 61, 62 and 63 (or a sequence at least 95% identical to any one of the same). In one embodiment the heavy chain is SEQ ID NO: 59 (or a sequence at least 95% identical thereto) and the light chain is independently selected from SEQ ID NO: 61, 62 and 63 (or a sequence at least 95% identical to any one of the same). In one embodiment the heavy chain is SEQ ID NO: 60 (or a sequence at least 95% identical thereto) and the light chain is independently selected from SEQ ID NO: 61, 62 and 63 (or a sequence at least 95% identical to any one of the same). In one embodiment the heavy chain is SEQ ID NO: 58 or 60 (or a sequence at least 95% identical to any one of the same) and a light chain with the sequence shown in SEQ ID NO: 61 (or a sequence at least 95% identical thereto). In one embodiment the heavy chain is SEQ ID NO: 58 (or a sequence at least 95% identical to any one of the same) and a light chain with the sequence shown in SEQ ID NO: 61 (or a sequence at least 95% identical thereto). In one embodiment the heavy chain is SEQ ID NO: 60 (or a sequence at least 95% identical to any one of the same) and a light chain with the sequence shown in SEQ ID NO: 61 (or a sequence at least 95% identical thereto).

In one embodiment the anti-IL13R antibody or antigen binding fragment thereof comprises a heavy chain constant region comprising the sequence EEQFNSTYR (SEQ ID NO: 64).

In one embodiment the anti-IL13R antibody is eblasakimab.

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have the exact same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human. Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425, 5,661,016, 5,770,429, EP 0438474 and EP0463151.

Constant region as employed herein is intended to refer to the constant region portion located between two variable domains, for example non-cognate variable domains, in the heavy chain. Thus, the presently disclosed anti-IL13R antibody may comprise one or more constant regions, such as a naturally occurring constant domain or a derivate of a naturally occurring domain.

A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53; Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO03031581. Alternatively, where the effector molecule is a protein or polypeptide the linkage may be achieved using recombinant DNA procedures, for example as described in WO86/01533 and EP0392745.

The term effector molecule as used herein includes, for example, biologically active proteins, for example enzymes, other antibody or antibody fragments, synthetic or naturally occurring polymers, nucleic acids and fragments thereof e.g., DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof. Specific naturally occurring polymers include lactose, amylose, dextran, glycogen, or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000 Da to about 40000 Da.

In one example antibodies for use in the present invention are attached to poly(ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

### Formulations

The present disclosure also provides composition and/or formulations comprising one or more anti-IL13R antibodies or antigen binding fragments thereof as defined above, and a pharmaceutically acceptable excipient, diluent or carrier.

In one embodiment at least 80%, such as 81, 82, 83, 84, 85 or 86% of the N-glycans in the composition and/or formulation are G0F.

In one embodiment less than 0.5 to 1%, such as 0.7% of the N-glycans are G0-N.

In one embodiment 3.5 to 4.5%, such as 3.7% or 3.8% of the N-glycans are G0F-N.

In one embodiment 1 to 2.5%, such as 1.5% or 1.9% of the N-glycans are G0.

In one embodiment 0.5 to 1.5%, such as 1.1% of the N-glycans are M5.

In one embodiment 3 to 4.5%, such as 3.5% or 4.1% of the N-glycans are G1F.

In one embodiment 3.5 to 5%, such as 3.7% or 4.3% of the N-glycans are G1F′.

In one embodiment 0.3 to 1%, such as 0.5% or 0.8% of the N-glycans are G2F.

In one embodiment the formulations of the present disclosure have a viscosity in the range of 10 to 30, such as 11, 12, 13, 14, 15, 16, 17, 18, 19, 20, 21, 22, 23, 24, 25, 26, 27, 28, 29 or 30 cP (centipoise), such as 20 cP, for example at ambient temperature. Surprisingly, the viscosity of the formations of the present disclosure are relatively low even at high concentrations of antibody.

In one embodiment, the viscosity is measured using a viscometer, such as rotational viscometer, an electromagnetically spinning-sphere (EMS) viscometer, or a Stabinger viscometer. In one embodiment the viscosity is measured using a rheometer, such as shear rheometer, dynamic shear rheometer, an extensional rheometer, a capillary rheometer. In one embodiment the viscosity is measured using a Kinexus-ultra+ rheometer (Netzsch).

In one embodiment the osmolarity of the formulation is in the range 350 to 450 mOsmo/kg, such as 390 to 430 mOsmo/kg, in particular 410+/−5 mOsmo/kg.

In one embodiment the formulation comprises 150 to 210 mg/ml or an anti-IL13R antibody, for example 150 to 175 mg/ml, such as 150, 155, 160, 165, 170, 175 mg/ml. In embodiment, the formulation comprises 175 mg/ml to 210 mg/ml, such as 175, 180, 185, 190, 195, 200, 205 or 210 mg/ml. In one embodiment the formulation comprises 150 mg/ml of anti-IL13R antibody. In another embodiment, the formulation comprises 175 mg/ml of anti-IL13R antibody. In one embodiment, the formulation comprises 200 mg/ml.

In one embodiment, the formulation comprises 170 to 260 mM of arginine, for example 170, 175, 180, 185, 190, 195, 200, 205, 210, 215, 220, 225, 230, 235, 240, 245, 250 or 260 mM. In one embodiment, the formulation comprises 150 mM, 175 mM, 200 mM or 250 mM arginine. In one embodiment the formulation comprises 150 mM arginine. In one embodiment the formulation comprises 175 mM arginine. In one embodiment the formulation comprises 200 mM arginine. In one embodiment the formulation comprises 250 mM arginine.

In one embodiment the arginine is Arg-HCl. In another embodiment, the arginine is Arg-Glu. In one embodiment arginine is L-arginine. Hence, in one embodiment the formulation comprises 150 mM Arg-HCl. In one embodiment the formulation comprises 175 mM Arg-HCl. In one embodiment the formulation comprises 200 mM Arg-HCl. In one embodiment the formulation comprises 250 mM Arg-HCl.

In one embodiment the formulation comprises 20 to 50 mM histidine buffer, for example 20, 25, 30, 35, 40, 45 or 50 mM, such as 20 mM or 50 mM histidine buffer. In one embodiment the formulation comprises 20 mM histidine buffer. In another embodiment the formulation comprises 50 mM histidine buffer.

In one embodiment the formulation comprises 0.01-0.03% of a non-ionic surfactant, such as 0.01, 0.015, 0.02, 0.025 or 0.030%, in particular 0.02%. In one embodiment the formulation comprises 0.01-0.03%, such as 0.01, 0.015, 0.02, 0.025 or 0.030%, in particular 0.02% volume per volume (v/v) of a non-ionic surfactant. In one embodiment the formulation comprises 0.01-0.03%, such as 0.01, 0.015, 0.02, 0.025 or 0.030%, in particular 0.02% weight per volume (w/v) of a non-ionic surfactant. In one embodiment the formulation comprises 0.01-0.03%, such as 0.01, 0.015, 0.02, 0.025 or 0.030%, in particular 0.02% weight per weight (w/w) of a non-ionic surfactant. In one embodiment the formulation comprises 0.02% w/w of a non-ionic surfactant.

In one embodiment the non-ionic surfactant is polysorbate, such as polysorbate 20, 40, 60, or 80. In one embodiment the non-ionic surfactant is polysorbate 20. Thus, in one embodiment the formulation comprises 0.01-0.03%, such as 0.02% polysorbate 20 (for example as % w/w, % w/v, % v/w or % v/v). In one embodiment the formulation comprises 0.02% w/w polysorbate 20.

In one embodiment wherein the pH of the formulation is in the range 6.0 to 7.0, such as 6.0, 6.1, 6.2, 6.3, 6.4, 6.5, 6.6, 6.7, 6.8, 6.9 or 7.0. In one embodiment the pH is 6.0, 6.5 or 7.0. In one embodiment, the pH is 6.5.

In one embodiment the formulation further comprises phenylalanine, such as 45 to 90 mM phenylalanine, for example 45, 50, 55, 60, 65, 70, 75, 80, 85 or 90 mM. In one embodiment the formulation comprises 50, 75 or 80 mM phenylalanine. Thus, in one embodiment the formulation comprises 50 mM phenylalanine. In one embodiment the formulation comprises 75 mM phenylalanine. In one embodiment the formulation comprises 80 mM phenylalanine.

In one embodiment the formulation further comprises CaCl₂), for example 10, 20, 30, 40, 50 or 60 mM CaCl₂). In one embodiment the formulation comprises 50 mM CaCl₂).

In one embodiment the formulation further comprises 50 to 200 mM of a sugar, such as 50, 60, 70, 80, 90, 100, 110, 120, 130, 140, 150, 160, 170, 180, 190 or 200 mM of a sugar. In one embodiment the formulation comprises 180 mM of a sugar. In one embodiment the sugar is selected from mannitol, sorbitol, dextrose, galactose, fructose, lactose, trehalose and sucrose. In one embodiment the sugar is sucrose. Thus in one embodiment the formulation comprises 180 mM sucrose. In one embodiment the formulation does not comprise a sugar.

In one embodiment certain formulations of the present disclosure have 1% or less protein aggregation, for example when stored for 90 days at temperature in the range 2 to 25° C.

The presently disclosed anti-IL13R antibody formulation is particularly suitable for stable long-term storage of the anti-IL13R antibody.

In one embodiment the formulation is stored at a temperature in the range 2 to 8° C., such as 2, 3, 4, 5, 6, 7 or 8° C., such as 4° C.

In one embodiment there is provided a parenteral formulation (in particular a liquid formulation) for example for infusion or injection. In one embodiment there is provided liquid parenteral formulation as a concentrate for dilution with a liquid for injection, such as glucose, saline or water for injection. In one embodiment the liquid parenteral formulation is provided in a final concentration for administration without dilution, for example for injection or for infusion.

### Treatment

The anti-IL13R antibody or binding fragment thereof or formulation thereof according to the present disclosure may be used for treatment or in the manufacture of a medicament. For example, the disclosed anti anti-IL13R antibody or binding fragment thereof or formulation thereof is suitable for use in treating an inflammatory disorder, such as chronic inflammation, or an autoimmune disease.

The inflammatory condition or disorder, may, for example be selected from the group comprising or consisting of arthritis such as rheumatoid arthritis, asthma such as severe asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, Lyme disease, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenia purpura, Meniere's disease, pemphigus, primary biliary cirrhosis, sarcoidosis, scleroderma, Wegener's granulomatosis, other autoimmune disorders, pancreatitis, trauma (surgery), graft-versus-host disease, transplant rejection, heart disease including ischaemic diseases (such as myocardial infarction as well as atherosclerosis), intravascular coagulation, bone resorption, osteoporosis, osteoarthritis, periodontitis, hypochlorhydia and cancer, including breast cancer, lung cancer, gastric cancer, ovarian cancer, hepatocellular cancer, colon cancer, pancreatic cancer, esophageal cancer, head & neck cancer, kidney, and cancer, in particular renal cell carcinoma, prostate cancer, liver cancer, melanoma, sarcoma, myeloma, neuroblastoma, placental choriocarcinoma, cervical cancer, and thyroid cancer, and the metastatic forms thereof.

In one embodiment the antibody or antigen-binding fragment thereof or formulation, according to the present disclosure is employed for the treatment of a chronic inflammatory condition wherein the condition associated with inappropriate inflammation. Such conditions include, but are not limited to, rheumatoid arthritis (RA), autoimmune conditions, inflammatory bowel diseases, non-healing wounds, multiple sclerosis, cancer, atherosclerosis, sjogrens disease, diabetes, lupus erythrematosus (including systemic lupus erythrematosus), asthma, fibrotic diseases (including liver cirrhosis), pulmonary fibrosis, and UV damage and psoriasis.

Chronic inflammation is a debilitating and serious condition associated with many of the above diseases and is characterised by persistent inflammation at a site of infection or injury, or persistent inflammation of an unknown origin, or in relation to altered immune responses such as in autoimmune disease.

Thus, in one embodiment the antibody or antigen-binding fragment, formulation or method according to the present disclosure is employed in the treatment of a chronic inflammatory condition wherein the condition is associated with any condition associated with inappropriate inflammation. Such conditions include, but are not limited to, rheumatoid arthritis (RA), autoimmune conditions, inflammatory bowel diseases, non-healing wounds, multiple sclerosis, cancer, atherosclerosis, Sjogrens disease, diabetes, lupus erythrematosus (including systemic lupus erythrematosus), asthma, fibrotic diseases (including liver cirrhosis), pulmonary fibrosis, UV damage and psoriasis.

In one embodiment the antibody or antigen-binding fragment thereof, formulation or method according to the present disclosure is employed in the treatment of a condition selected from axial spondyloarthropathy, primary biliary cholangitis, and allergy, for example a food allergy such as a peanut allergy, or a pollen allergy.

In one embodiment the inflammatory disorder or autoimmune disease is selected from the group comprising: fibrosis (including pulmonary fibrosis, such as cystic fibrosis, iodiopathic pulmonary fibrosis, progressive massive fibrosis; liver fibrosis, such as cirrhosis; heart disease, such as atrial fibrosis, endomyocardial fibrosis, old myocardial infarction; arthrofibrosis; Dupuytren's contracture; keloid fibrosis; mediastinal fibrosis; myelofibrosis; nephrogenic systemic fibrosis; retroperitoneal fibrosis; and scleroderma) Hodgkin's disease, ulcerative colitis, Chron's disease, atopic dermatitis, eosinophilic esophagitis, allergic rhinitis, asthma and chronic pulmonary disease (including chronic obstructive pulmonary disease).

In patients with cancer, such as breast cancer, cancer related lymphedema (BCRL), the formulation of the present disclosure may prevent lymphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lymphedema after lymphatic injury is associated with tissue inflammation, the infiltration of CD4-positive cells and their differentiation to the type 2 helper T-cell (Th2) phenotype. Th2 cells produce IL-4 and IL-13 that play a key role in the development of lymphedema-associated symptoms as well as other Th2-mediated diseases.

In one embodiment the antibody, binding fragment or formulation of the present disclosure is used for the treatment of asthma or is used for the manufacture of a medicament for the treatment of the same. In one embodiment the antibody, binding fragment or formulation of the present disclosure is used for the treatment of dermatitis (such as atopic dermatitis) or is used for the manufacture of a medicament for the treatment of the same. In one embodiment the antibody, binding fragment or formulation of the present disclosure is used for the treatment of Psoriasis or is used for the manufacture of a medicament for the treatment of the same.

In one embodiment the antibody, binding fragment or formulation of the present disclosure is employed as a monotherapy.

In one embodiment the formulation herein is administered in combination with another therapy, for example an anti-inflammatory agent, such as a non-steroidal anti-inflammatory and/or a steroid (e.g. prednisolone or prednisolone).

“In combination” as employed herein is intended to encompass where the anti-IL13R antibody is administered before, concurrently with another therapy.

Therapeutic dose as employed herein refers to the amount of the anti-IL13R antibody, such as eblasakimab that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment a formulation according to the present disclosure (including a formulation comprising same) is administered monthly, for example in a treatment cycle or as maintenance therapy.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Individual elements from the reagents, method steps and processes recited herein, including in the examples, may form the basis of features in the claims even if isolates from other parameters.

Technical references such as patents and applications are incorporated herein by reference.

Any embodiments specifically and explicitly recited herein may form the basis of a disclaimer either alone or in combination with one or more further embodiments.

Subject headings herein are employed to divide the document into sections and are not intended to be used to construe the meaning of the disclosure provided herein.

The present invention is further described by way of illustration only in the following examples.

### BRIEF DESCRIPTION OF FIGURES

FIG. 1 shows a flowchart summarising the cell culture process.

FIG. 2A shows the MS/MS spectrum of T26 peptide with G0F

FIG. 2B shows the MS/MS spectrum of T26 peptide with G0F

FIG. 2C shows the MS/MS spectrum of Deglycosylated T26 peptide

FIG. 2D shows the MS/MS spectrum of the glycosylated T26 peptide.

FIG. 3 shows a summary table of the oligosaccharide composition of eblasakimab.

FIG. 4A shows a table of the oligosaccharide composition of eblasakimab across the various batches tested.

FIG. 4B shows ms/ms spectrum of oligosaccharide composition of eblasakimab across the various batches tested.

FIG. 5 shows an overview of symbols and abbreviation of the oligosaccharide structures identified by oligosaccharide mapping analysis.

### ABBREVIATIONS

MCB: master cell bank
WFI: water for injection
PBS: phosphate buffered saline
IPA: isopropanol alcohol
CFU: colony forming unit
CCF: cell culture fluid
PCR: polymerase chain reaction
HPLC: high performance liquid chromatograph
WCB: working cell bank
BSC: biological safety cabinet
CHO: Chinese Hamster Ovary
VCD: viable cell density
IPC: in-process control
HCCF: harvest cell culture fluid
TCM: transmission electron microscope

### EXAMPLES

### Example 1—Production of ASLAN004

### 1. Cell Line Details

Cell type: CHO-M
Cell bank history: Cell seed—cells from Selexis with 6×10⁶ cells per vial, Lot: 20161028AM11; P2/day 3
Master cell bank (MCB): manufactured from cell seed with 2.0×10⁷ cells per vial, Lot: 01-104-2221-01 (30 Mar. 2017)
Working cell bank (WCB): manufactured from MCB with 2.0×10⁷ cells per vial, Lot: 01-105-2221-01 (4 May 2017)

### 2. Preparation of Medium/Solutions

### Selective Medium (Total Volume 4 L)

Preparation procedure:
1. 3 L water for injection (WFI) was transferred into 5 L beaker and agitated.
2. BalanCD CHO Growth A, L-glutamine, Sodium bicarbonate was added in sequence into the beaker then WFI was added to 4 L.
3. The solution was mixed for at least 30 minutes but not more than 2 hours
4. The pH was measured via pH meter. The pH was adjusted to within 7.0±0.2 by 1N sodium hydroxide solutionif necessary
5. Puromycin and Hygromycin stock solution was added into the beaker
6. The medium was mixed at least 10 minutes. pH, L-glutamine and Osmolality was measured to ensure these were within controlled range
7. The medium was filtered in Biological Safety Cabinet (BSC) via cup filter to a 1 L storage bottle
Storage and expiration (sterile):
   Expiry 30 days if stored in dark at 2-8° C.
Control Parameters:
   1. pH (via pH meter): 6.8-7.2
   2. L-glutamine (via NOVA BioProfile FLEX): 5.0-7.0 mM
   3. Osmolality (via NOVA BioProfile FLEX): 290-330 mOsm/Kg
In-process monitor:

### Bioburden Test and Endotoxin Test

### Production Medium (Total Volume: 60 L/330 L)

Preparation procedure:
1. WFI was transferred to 80-90% of final volume in an appropriately sized mixer and agitated.
2. BalanCD CHO Growth A Medium powder, L-glutamine, and Sodium bicarbonate were added into the mixer in sequence.
3. The solution was mixed for at least 60 minutes but not more than 2 hours.
4. The pH was measured via pH meter. The pH was adjusted within 7.0±0.2 by 10N sodium hydroxidesolution if necessary
5. WFI was added to final weight: 60.3 Kg or 331.65 Kg (medium density: 1.005 g/ml)
6. The medium was mixed for additional 10 minutes but not more than 20 minutes
7. The pH was verified to be within 6.8-7.2 by pH meter.
8. L-glutamine and Osmolality was determined with NOVA BioProfile FLEX and verified to be within the controlled range
9. The medium was filtered using a Sartopore 2 filter
10. Storage and expiration (sterile):
   Expiry 15 days if stored in dark at 2-8° C.
Control parameters:
1. pH (via pH meter): 6.8-7.2
2. L-glutamine (via NOVA BioProfile FLEX): 5.0-7.0 mM
3. Osmolality (via NOVA BioProfile FLEX): 290-330 mOsm/Kg.

In-process monitor:

### Bioburden Test and Endotoxin Test

### 1 M Sodium Carbonate Solution (Total Volume 5 L).

Preparation procedure:
   1. WFI was transferred to 80% of final volume in an appropriately sized mixing tank and agitated
   2. Sodium carbonate powder was added.
   3. WFI was added to final weight: 5.5 Kg (solution density: 1.1 g/mL)
   4. The solution was mixed for at least 30 minutes
   5. The pH was measured via pH meter and the pH verified to be within controlled range
   6. The medium was filtered via Sartopore 2 Filter
Storage and expiration (sterile):
Expiry 1 year stored at room temperature.
Control parameters:
   pH (via pH meter): 11-13

### 20% Lactic Acid (Total Volume 60 L)

Preparation procedure:
1. WFI was transferred to 70% of final volume in an appropriately sized mixing tank and agitated.
2. 12 L Lactic acid was added
3. WFI was added to final weight: 60 Kg (solution density: 1.0 g/mL)
4. The solution was mixed for at least 30 minutes
5. The medium was filtered via Sartopore 2 Filter
Storage and expiration (sterile):
Expiry 30 days stored at room temperature
Control Parameter: N/A
   30% Glucose Solution (Total volume 20 L).
Preparation procedure:
1. WFI was transferred to 60% of final volume in an appropriately sized mixing tank and agitated
2. D(+)-Glucose anhydrous powder was added
3. WFI was added to final weight 22.24 Kg (solution density: 1.112 Kg/L).
4. The solution was mixed for at least 2 hours until the solution was clear
5. 20 ul solution was taken and mixed with 980 ul 1×PBS for 50-fold dilution. The glucose concentration was analysed with NOVA and verified to be within the controlled range
6. The glucose solution was filtered via Millipak 60 filter.
Storage and expiration (sterile):
Expiry 1 year stored at room temperature
Control parameters:

### Glucose Concentration (Via NOVA BioProfile FLEX)

### Cell Boost 7a

Preparation procedure:
1. WFI was transferred to 75% of final volume in an appropriately sized mixer and agitated
2. Cell Boost 7a powder (Hyclone™) was added into the mixer
3. The solution was mixed for at least 30 minutes but not more than 1 hour
4. Sodium Hydroxide was added
5. The solution was mixed at least 60 minutes but not more than 2 hours.
6. WFI was added to final weight 103 Kg (solution density: 1.03 g/ml)
7. The solution was mixed for an additional 10 minutes but not more than 20 minutes.
8. The pH was measured via pH meter and adjusted to within 6.7±0.1 by adding 10N sodium hydroxide solution if necessary
9. 1 ml of the solution was mixed with 4 ml WFI for 5-fold dilution and the osmolality was measured via NOVA BioProfile FLEX. Osmolality was verified to be within the controlled range,
10. The solution was filtered via Opticap XL05 SHF filter.
Storage and expiration (sterile):
Expiry 14 days if stored in dark at 2-8° C.
Control parameters:
pH (via pH meter): 6.7±0.1
Osmolality (via NOVA BioProfile FLEX): 247-303 mOsm/Kg.
In-process monitor:

### Bioburden Test and Endotoxin Test

### Cell Boost 7b

Preparation procedure:
1. WFI was transferred to 80% of final volume in an appropriately sized mixer and agitated
2. Cell Boost 7b powder (Hyclone™) was added into the mixer
3. The solution was mixed for at least 30 minutes but not more than 60 minutes
4. Sodium Hydroxide was added
5. The solution was mixed for at least 60 minutes but not more than 2 hours
6. The pH was measured via pH meter and adjusted to within 11.0-11.4 by adding 10N sodium hydroxide solution if necessary
7. The solution was mixed for at least 60 minutes but not more than 2 hours.
8. WFI was added to final weight: 10.92 Kg (medium density: 1.04 g/ml)
9. The solution was mixed for an additional 10 minutes but not more than 20 minutes.
10. The pH was verified to be within 11.0-11.4 by pH meter.
11. 1 ml of the solution was taken and mixed with 4 ml WFI for 5-fold dilution. The osmolality was measured via NOVA BioProfile FLEX and verified to be within controlled range.
12. The solution was filtered via Sartopore 2 filter.
Storage and expiration (sterile):
Expiry 14 days if stored in dark at 2-8° C.
Control parameters:
pH (via pH meter): 11.0-11.4
Osmolality (via NOVA BioProfile FLEX): 218-266 mOsm/Kg.
In-process monitor:

### Bioburden Test and Endotoxin Test

### 1×PBS (Phosphate Buffered Saline)

Preparation procedure:
1. 10×PBS starting volume was diluted to 10% of final volume (assume that the density is 1 Kg/L) using WFI.
2. The solution was agitated at 150 rpm and mixed at least 10 minutes
3. The 1×PBS was ready for use.
   Storage and expiration (non-sterile): Expiry 1 day stored at room temperature

### 3. ASLAN004 Manufacturing Procedure

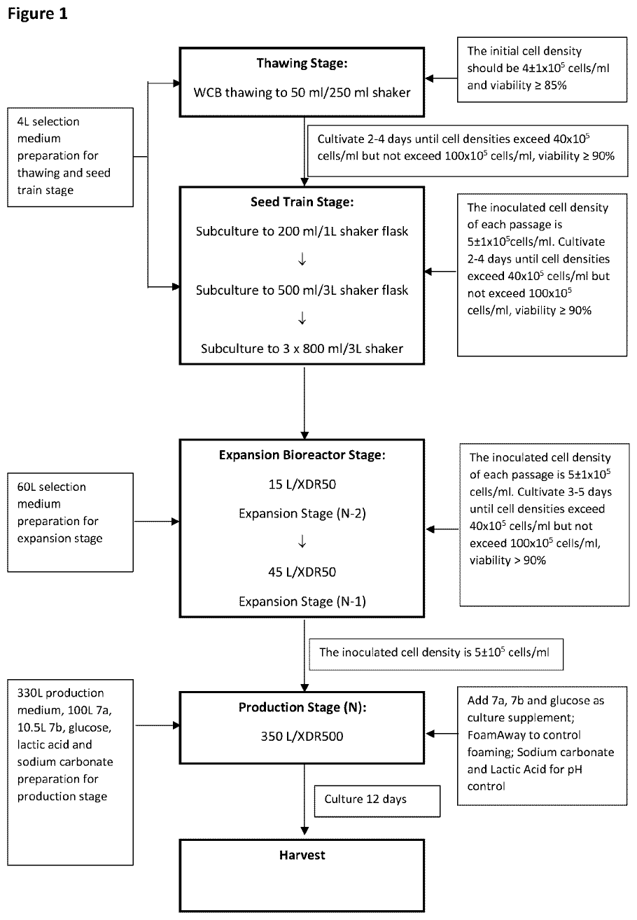

An overview of the cell culture process is shown in FIG. 1.

### 3.1 Thaw Stage Operating Parameters

1. The selective medium was pre-warmed in a 37° C. water bath for at least 20 minutes.
2. A 50 ml centrifuge tube containing 7.5 ml 70% Isopropanol alcohol (IPA) was pre-warmed in a 37° C. water bath for at least 20 minutes.
3. The pre-warmed medium was transferred to the biological safety cabinet (BSC) in the grade C clean room. 49 ml selective medium was added into one 250 ml shaker flask in BSC.
4. The cryovial containing the cells was removed from the liquid nitrogen storage container and immediately placed into the pre-warmed 50 ml centrifuge tube at 37° C. water bath for 5 minutes.
5. The vial was removed from the 50 ml centrifuge tube, dried with a paper towel, the vial label peeled off and pasted into the batch record. Next the outer surface of the vial was cleaned with IPA and the vial was transferred into a grade C clean room and placed in a BSC.
6. The entire cell fluid was aspirated from the vial and then re-suspended in fresh selective medium in the 250 ml shaker flask.
7. The flask was placed into the incubator, and the culture conditions were set as follows:

| | | |
|---|---|---|
| Temperature | 37° | C. |
| CO₂ concentration | 5% | (v/v) |

| | |
|---|---|
| Agitation speed | 130 rpm 50 mm |
| | orbital throw |

8. After 5 minutes, a 1 ml sample was taken for cell count via NOVA-BioProfile FLEX or Microscope. The target initial viable cell density and cell viability after thaw is 4±1×10⁵ cells/ml and ≥85%.
9. After incubating the cells for 2-4 days, the cells were expanded to one 1 L shake flask when viable cell density (VCD) reached 40×10⁵<VCD≤100×10⁵ viable cells/ml and viability ≥90%.
10. If the viable cell density was lower than 40×10⁵ cells/ml, the cells were sub-cultured to 1×250 ml shaker flask with working volume 50 ml.

### 3.2 Seed Train Stage Operating Parameters

1. Control parameters of shaking incubator:

| | | |
|---|---|---|
| Temperature | 37° | C. |
| CO₂ concentration | 5% | (v/v) |

| | |
|---|---|
| Agitation speed | 130 rpm, 50 mm |
| | orbital throw |

2. Shaker flask size and working volumes:

| Working Volumes | Shaker Flask Size |
|---|---|
| 50 ml | 250 ml SF |
| 200 ml | 1 L SF |
| 500 ml/800 ml | 3 L SF |

3. Cell density subculture criteria:

| | |
|---|---|
| Seeding | 5 ± 1 × 10⁵ cells/ml and viability ≥90% |
| Subculture | 40 × 10⁵ < VCD ≤ 100 × 10⁵ viable |
| | cells/ml and viability ≥90% |

4. Cells were passaged in the shake flask every 2-4 days in the Selection Medium.
5. When cell density reached 40×10⁵<VCD≤100×10⁵ viable cells/ml and the culture viability ≥90%, the culture was forward passaged to the bioreactor stage.

### 3.3 Expansion Stage Operating Parameters

1. Initial conditions for the Expansion Stage

| | |
|---|---|
| Bioreactor Scale | 50 L |
| Medium | Production Medium |
| Working Volume | N-2: 15 L |
| | N-1: 45 L |

2. Temperature: 37° C.
3. pH:
   Set point: 7.00±0.20.
   pH control: automated control by 1M sodium carbonate and carbon dioxide
   Look up table setting:

| | | |
|---|---|---|
| pH control | AE-01 → Split Range 1 | SR1 Low → MFC3 |
| | | SR1 UP → Pump 1 |

Offset adjustment procedure: when pH probe readings differ to the offline (Nova) reading by >0.05 pH unit during culture, a one-point calibration was performed to adjust back to the offline reading.
4. DO:
   Set point: 50%
   DO control: automated control by cascade sparging strategy with air until maximum limit and then supplemented additionally with oxygen
   Operating parameter of Look up table for DO control:

| | | |
|---|---|---|
| DO control | AE-02 → look up table 1 | look up table 1 → MFC1 |
| | AE-02 → look up table 2 | look up table 2 → MFC2 |

| | |
|---|---|
| Look up table 1 | Look up table 2 |

| | In | Out | | In | Out |
|---|---|---|---|---|---|
| Row0 | 0.0 | 1.0 | Row0 | 0.0 | 0.0 |
| Row 1 | 100.0 | 1.0 | Row 1 | 29.0 | 0.0 |
| | | | Row2 | 30.0 | 0.2 |
| | | | Row3 | 50.0 | 2.5 |
| | | | Row4 | 100.0 | 5.0 |

5. Air-head space flow rate: 0.5 LPM
6. Sparged Air and 0₂: Use 1 mm sparger port.
7. pCO₂: maintained below 100 mm Hg by controlling air flow.
8. Agitation rate: N−2 Stage: 60 rpm/N−1 Stage: 75 rpm
9. Antifoam: antifoam added if foam thickness was more than 5 cm.
10. Cell density subculture criteria:

| | |
|---|---|
| Seeding | 5 ± 1 × 10⁵ cells/ml and viability ≥90% |
| Culture Duration | 3-5 days |
| Subculture | 40 × 10⁵ ≤ VCD ≤ 100 × 10⁵ viable |
| | cells/ml and viability >90% |

11. Sampling: The cell culture was sampled daily to analyze gas profile, nutrient concentration and cell growth profile via NOVA Bioprofile FLEX.

### 3.4 Production Stage Operating Parameters in Bioreactor

1. Initial conditions for the Production Stage

| | |
|---|---|
| Bioreactor Scale | 500 L |
| Medium | Production Medium |
| Initial Working Volume | 350 L |

2. Temperature: Day 0 to Day 5:37° C.; Day 6 to Day 12:33° C.
3. pH:
   Set point: 7.00±0.05.
   pH control: From Day 0 to Day 3, automated control by 1M sodium carbonate and carbon dioxide. From Day 3 to harvest, automated control by 1M sodium carbonate and 20% (v/v) lactic acid.
   Look up table setting:

From Day 0 to Day 3

| | | |
|---|---|---|
| pH control | AE-01 → Split Range 1 | SR1 Low → MFC3 |
| | | SR1 UP → Pump1 |

From Day 3 to Harvest

| | | |
|---|---|---|
| pH control | AE-01 → Split Range 1 | SR1 Low → Pump 2 |
| | | SR1 UP → Pump1 |

Offset adjustment procedure: when pH probe readings differ from the offline (Nova) reading by >0.05 pH unit during culture, a one-point calibration was performed to adjust back to the offline reading.
4. Dissolved Oxygen (DO):
   Set point: 50%
   DO control: automated control by cascade sparging strategy with air and oxygen
   Operating parameter of look up table for DO control:

| | | |
|---|---|---|
| Do control | AE-02 → Look up table 1 | Look up table 1 → MFC1 |
| | AE-02 → Look up table 2 | Look up table 2 → MFC6 |
| | AE-02 → Look up table 3 | Look up table 3 → MFC2 |

| Look up table 1 | | | Look up table 2 | | | Look up table 3 | | |
|---|---|---|---|---|---|---|---|---|
| | In | Out | | In | Out | | In | Out |
| Row 0 | 0.0 | 3.0 | Row 0 | 0.0 | 0.0 | Row 0 | 0.0 | 0.0 |
| Row 1 | 100.0 | 3.0 | Row 1 | 15.0 | 0.0 | Row 0 | 70.0 | 0.0 |
| | | | Row 2 | 70.0 | 20.0 | Row 0 | 100.0 | 10.0 |
| | | | Row 3 | 100.0 | 20.0 | | | |

5. Air-head space flow rate: 2.0 LPM
6. Sparged Air and 0₂: Use 1 mm spager port.
7. pCO₂: maintained below 100 mm Hg by controlling air flow. If the pCO₂ exceeds 100 mmHg, 2 mm sparger Tee was opened to aerate additional air for CO₂ stripping. However, pCO₂ was not considered to be a mandatory control parameter depending on the culture conditions.
8. Agitation rate: 115 rpm
9. Antifoam: add antifoam if foam thickness is more than approximately 5 cm.
10. Cell density subculture criteria:

| | |
|---|---|
| Inoculation | 5 ± 1 × 10⁵ cells/ml and viability ≥90% |
| Culture Length | 12 days |

11. Feeding strategy: Cell Boost 7a, Cell Boost 7b and 30% glucose solution were used as the feeding solution. After daily feeding (including glucose), the final NOVA glucose value should be 4 g/L.
   Cell Boost 7a: 10.5 Kg, 3% of initial working volume from Day 3 to Day 11
   Cell Boost 7b: 1.05 Kg, 0.3% of initial working volume from Day 3 to Day 11
   Glucose feed: when daily NOVA glucose value before feeding was below 4.0 g/L, glucose concentration was replenished to 4 g/L by adding 30% glucose solution according to the following formulation:

Kg of 300 g/L glucose added=((4.00−NOVA glucose value)×culture weight (kg)/300 g/L)×1.112
12. Sampling: The cell culture was sampled daily to analyze gas profile, nutrient concentration and cell growth profile via NOVA Bioprofile FLEX

### 3.5 Harvest Stage Operating Parameters in Bioreactor

1. Harvest filters

| | | | | | Recommended |
|---|---|---|---|---|---|
| | | | | Suggested | number of |
| | | | Loading | safety | filters when |
| Filter | | | capacity | margin | safety margin |
| location | Description | Vendor/Filter name | (L/m²) | (%) | is 50% |
| 1^{st} stage | Depth | Millipore/Millistak+ ® | 28 | 40-70 | 16 × C0HC |
| | filtration | HC Pod Depth filter, | | | |
| | | C0HC media series | | | |
| | | 1.1 m² | | | |
| 2^{nd} stage | Sterile | Millipore/Opticap ® | 438.6 | 40-70 | 3 × SHF XL10 |
| | filtration | Millipore Express ® | | | |
| | | SHF sterile 0.2 μm | | | |

2. Harvest process:
   Depth filter size: 17.6 m²
   Sterile filter size: 1.71 m²

1. Depth filter pre-flush
Step 1 - WFI pre-flush

| | |
|---|---|
| Equipment | Harvest Filtration Pump Skid |
| CV table | 50 |
| Amount of flush | 1000 L WFI |
| buffer/medium [L] | |

Step 2 - PBS pre-flush

| | |
|---|---|
| Equipment | Harvest Filtration Pump Skid |
| CV table | 50 |
| Amount of flush buffer/medium [L] | 160 L PBS |

2. Filtration

| Depth filtration | | Sterile filtration | | |
|---|---|---|---|---|
| control parameter | | control parameter | | |
| | Harvest | | 620 Bp | |
| | Filtration | | Peristaltic | |
| Equipment | Pump Skid | Equipment | Pump | |
| CV table | 10~20 | Flow rate | 10 | L/min |
| Pressure (psi) | <8-10 psi | Pressure (psi) | <12 | psi |

3. PBS Flush of filters at the end of harvest

| | Harvest | | 620 Bp | |
|---|---|---|---|---|
| | Filtration | | Peristaltic | |
| Equipment | Pump Skid | Equipment | Pump | |
| CV table | 20~30 | Flow rate | 10 | L/min |
| Amount | 160 L | Pressure (psi) | <12 | psi |

4. Air blow after flush

| | |
|---|---|
| Pressure (psi) | 5 |
| Time (min) | <30 |

3. Sampling, In-process control and acceptable criteria

| Buffer/Medium | | Acceptance | |
|---|---|---|---|
| Name | Test Item | Criteria | Sample Volume |
| Selective Medium | Bioburden (CFU/10 ml) | ≤1 CFU/10 ml | 25-50 ml |
| | Endotoxin (EU/ml) | ≤0.1 EU/ml | 2-10 ml |
| Production Medium | Bioburden (CFU/10 ml) | ≤1 CFU/10 ml | 25-50 ml |
| | Endotoxin (EU/ml) | ≤0.1 EU/ml | 1-10 ml |
| Cell Boost 7a | Bioburden (CFU/10 ml) | ≤1 CFU/10 ml | 25-50 ml |
| | Endotoxin (EU/ml) | ≤0.1 EU/ml | 2-10 ml |
| Cell Boost 7b | Bioburden (CFU/10 ml) | ≤1 CFU/10 ml | 25-50 ml |
| | Endotoxin (EU/ml) | ≤0.1 EU/ml | 2-10 ml |

In-process control (IPC)

| | | Acceptance | |
|---|---|---|---|
| IPC Sample Name | Test Item | Criteria | Sample Volume |
| Cell Culture Fluid | Bioburden (CFU/0.1 mL)^{(b)} | Report | 2-5 ml (sterile) |
| (CCF)^{(a)} | Mycoplasma (PCR)^{(c)} | Negative | ~2 ml |
| | Adventitious virus | Not detected | 40-40 ml x 2 tubes |
| | Mycoplasma (culture base) | Negative | 40-50 ml x 4 tubes |
| | | | and 2 ml x 2 |
| | | | eppendorfs |
| Harvest Cell | Bioburden (CFU/10 ml)^{(b)} | Report | 44-50 ml (sterile) |
| Culture Fluid | Endotoxin (EU/ml) | Report | 2-10 ml |
| (HCCF) | HPLC-Protein A^{(d)} | Report | 2-10 ml |

^{(a)}Sample was aliquoted at QC lab if required. The storage condition for CCF is ≤−20° C. after aliquoting.
^{(b)}For CCF Bioburden assay, the direct inoculation method was used. For the test of HCCF Bioburden assay, the filtration method was used.
^{(c)}Mycoplasma (PCR) test was performed on the CCF sampled two days prior to harvest (D-2).
^{(d)}In-Process Control (hold step)

In-process monitoring (IPM)

| | | Acceptance | |
|---|---|---|---|
| IPC Sample Name | Test Item | Criteria | Sample Volume |
| Cell Culture Fluid | HPLC protein^{(a)} | Report | 2-10 ml |
| (CCF)^{(a)} | Transmission Electron | Report | 10-15 ml |
| | Microscope (TEM) | | |
| Harvest Cell | HCP ELASA | Report | 2-5 ml |
| Culture Fluid | HCDNA | Report | 2-5 ml |
| (HCCF) | Retain sample | N/A | 10-15 ml |

^{(a)}CCF was filtered with disc filter prior to test

### Example 2—Identification of N-glycosylation Site

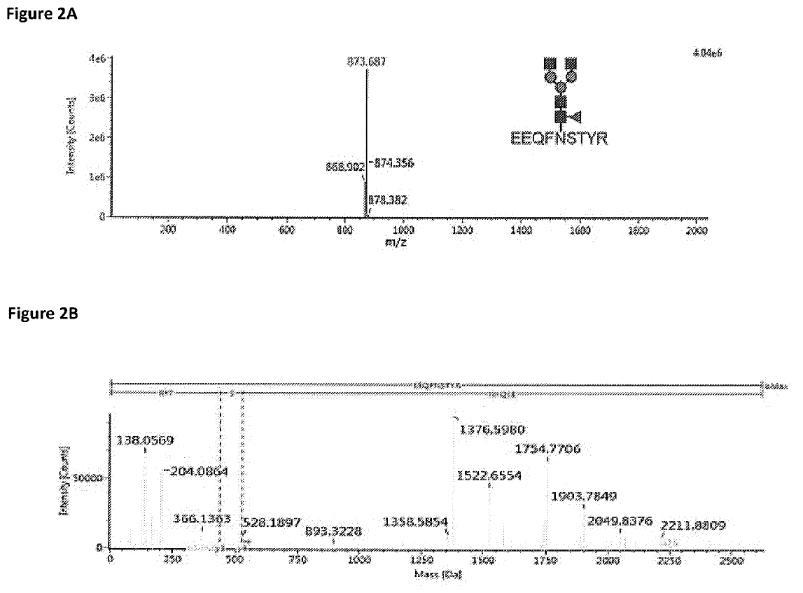

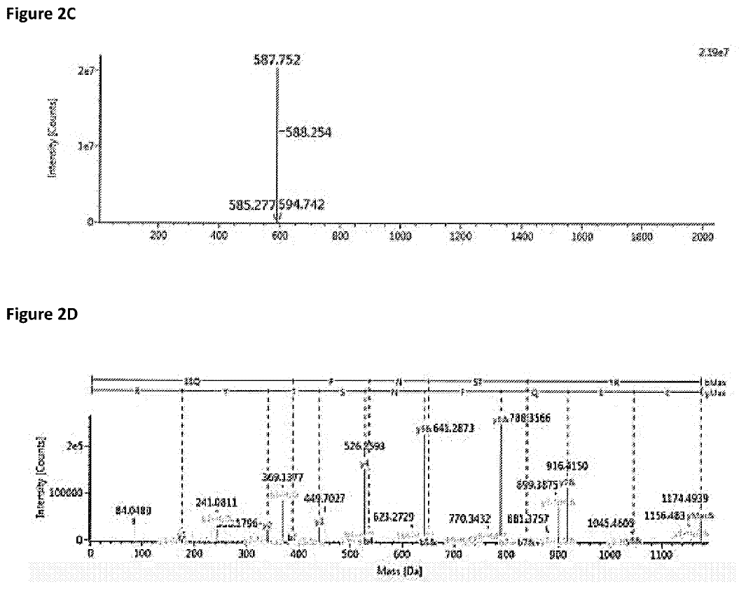

Site-specific N-glycosylation analysis was carried out on a reversed phase (RP) LC-MS/MS ion trap platform by identifying N295-glycopeptide (tryptic peptide T26) and deglycosylated T26 after PNGase F treatment. The conserved glycosylation motif N295-X-S/T is located in the heavy chain of ASLAN004. LC-MS/MS data of ASLAN004 Drug Substance batch Z22211701 presenting the tryptic peptide T26 and deglycosylated T26 is shown in FIGS. 2A to 2D.

The tryptic peptide T26 has a mass representative of the peptide, ₂₉₂EEQFNSTYR₂₉₉, plus a G0F glycan. PNGase F treatment then removes a mass corresponding to the G0F glycan removal and Asn-295 deamination, thus confirming Asn-295 as the glycosylation site. This corresponds to Asn-297 based on the EU numbering system for IgG molecules. This is also consistent with reference standard data from ASLAN004 Drug Substance batch ZT22211701.

### Example 3—N-Linked Oligosaccharide Mapping (Fluorescence Labeled Oligosaccharide Profile)

This method was developed to allow monitoring of the glycosylation pattern of ASLAN004. N-linked glycan analysis was performed using fluorescent tag methodology. In this method glycans were released from ASLAN004 using PNGaseF. Fluorescent labeling with 2-aminobenzamide (2-AB) was then carried out before detecting the labeled glycans by HPLC chromatographic separation with fluorescence detection. The glycan profile was determined by Comparison of ASLAN004 oligosaccharide elution patterns to those of known standards confirmed the presence of expected oligosaccharide species in the oligosaccharide map.

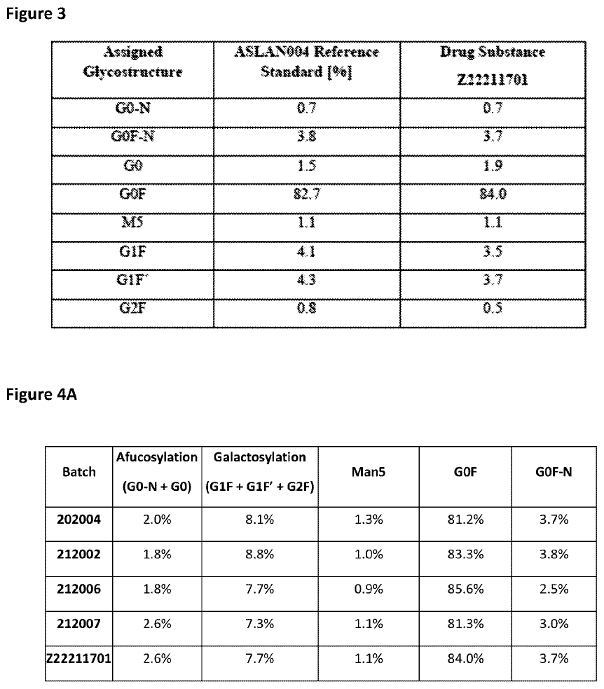

FIG. 3 shows the quantitative results for the reference standard and the GMP DS batch Z22211701 and reveals comparable levels of oligosaccharides between the GMP DS batch Z22211701 and the ASLAN004 reference standard (batch ZT22211701). The glycans present are expressed as a percentage of total area for the detected glycans, i.e. the % values represent the proportion of each N-glycan vs total N-glycans. The reference standard was established from a non-GMP batch, which followed GMP manufacture and was essentially the engineering batch. No changes were made to the manufacture procedures for ASLAN004 between the non-GMP and GMP batch.

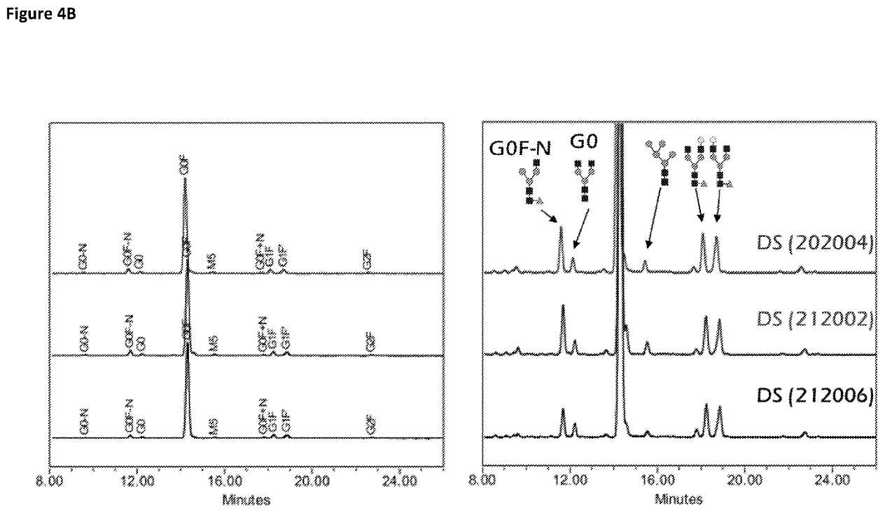

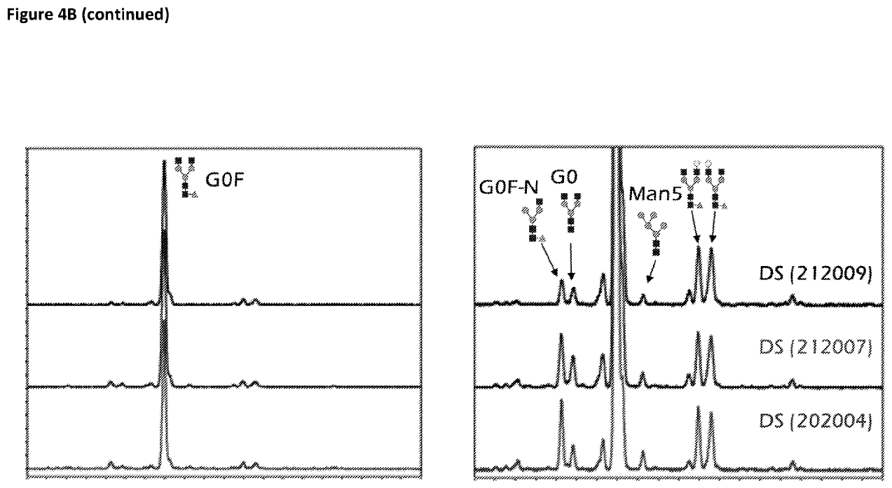

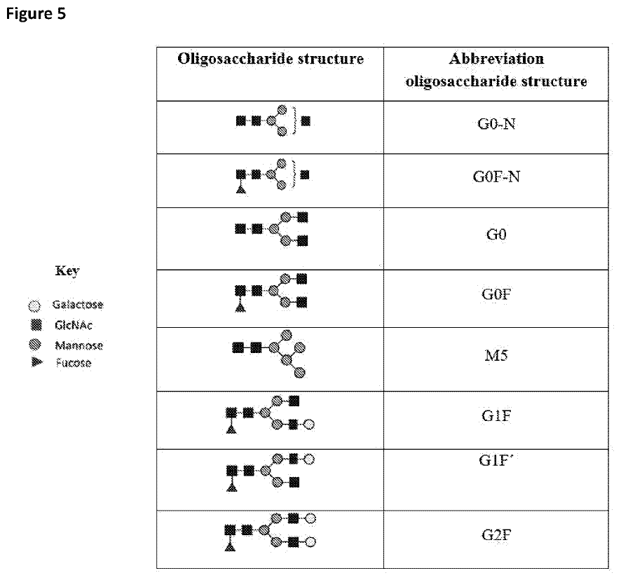

FIG. 4A shows the oligosaccharide composition of ASLAN004 across the various batches tested. FIG. 4B shows a sample of the MS/MS spectra across the batches tested. An overview of symbols and abbreviations of the oligosaccharide structures identified by oligosaccharide mapping analysis is provided in FIG. 5.

1.-34. (canceled)

35. A composition of an anti-IL-13R antibody manufactured in a mammalian cell line, comprising:
a. a VH CDR1 sequence as set forth in SEQ ID NO: 1, a VH CDR2; sequence as set forth in SEQ ID NO: 2, a VH CDR3 s set forth in SEQ ID NO: 10,
b. a VL CDR1 as set forth in SEQ ID NO: 31, a VL CDR2 sequence as set forth in SEQ ID NO: 32, and a VL CDR3 set forth in SEQ ID NO: 45;
c. a heavy chain constant region domain comprising the sequence EEQFNSTYR (SEQ ID NO: 64);
wherein the antibody is isotype IgG4 or IgG4 with a 241P mutation, and the N in SEQ ID NO: 64 is linked to a glycan (i.e. an N-glycan),
characterized in that the N-glycan population is at least 60% G0F, and afucosylated glycans are in the range 0 to 3%.

36. A composition according to claim 35, wherein 0.5 to 1.5% of the N-glycan population is M5.

37. A composition according to claim 35, wherein 6 to 10% of the N-glycan population are galactosylated glycans (such as G1F, G1F′ and/or G2F).

38. A composition according to claim 35, wherein the antibody is manufactured in a cell line genetically engineered to increase the extent of terminal sialic acid in glycoproteins expressed in the cells.

39. The composition according to claim 35, wherein the glycan comprises in the range 5 to 11 saccharides (sugar molecules), for example 5, 6, 7, 8, 9, 10 or 11, such as 6, 7, 8, 9 or 10, in particular 7 or 8.

40. The composition according to claim 35, wherein the glycan comprises 1 to 4 N-acetylglucosamine(s) (GlcAc), for example 1 to 4 N-acetylglucosamine, such as 1, 2, 3 or 4 N-acetylglucosamines, in particular 2 or 4.

41. The composition according to claim 35, wherein an N-acetylglucosamine is bonding to the N in SEQ ID NO: 64.

42. The composition according to claim 35, wherein the glycan comprises mannose, for example 1 to 5 mannose, such as 1, 2, 3, 4 or 5 mannose, more specifically 3 or 5 mannose, in particular 3 mannoses.

43. The composition according to claim 35, where the glycan further comprises galactose, for example 1 to 3 galactose, such as 1 or 2.

44. The composition according to claim 43, wherein galactose is the terminating saccharide free (unlinked) end of the glycan.

45. The composition according to claim 35, wherein the glycan comprises fucose, for example 1 to 2 fucose, such as 1.

46. The composition according to claim 45, wherein a fucose is linked to an N-acetylglucosamine, for example wherein the N-acetylglucosamine is bonding the N in SEQ ID NO: 64.

47. The composition according to claim 35, wherein the N-glycan does not comprise fucose and/or galactose.

48. The composition according to claim 35, wherein the glycans are selected from the group comprising:
i. G0-N, G0F-N, G0, G0F, M5, G1F, G1F′ and G2F;
ii. G0F-N, G0, G0F, M5, G1F and G1F′; or
iii. ISG0F-N, G0F, G1F and G1F′.

49. The composition according to claim 35, wherein the anti-IL13R antibody comprises a VH domain with a sequence shown in SEQ ID NO: 51 or a sequence at least 95% identical thereto, and a VL domain with a sequence shown in SEQ ID NO: 53 or a sequence at least 95% identical thereto.

50. A composition according to claim 35, wherein the composition is characterized by a population of glycans, such that G0F comprises in the range 80 to 90% of the population, such as 81, 82, 83, 84, 85 or 86%.

51. A composition according to claim 35, wherein the population of glycans comprises:
i. G0F-N 1 to 5% of the population, such as 2 to 4%, for example 2.5 to 3.9%, in particular 2.5, 3.0, 3.7 or 3.8%, such as 3.7%;
ii. G1F′ 1 to 5% of the population, such as 3.5 to 3.9%, in particular 3.7%.
iii. G1F, for example 1 to 5% of the population, such as 3.2 to 3.2%, in particular 3.5%.
iv. G0 1 to 3% of the population, such as 1.7 to 2.1%, in particular 1.9%.
v. M5 0.5 to 1.5% of the population, such as 0.9 to 1.3%, in particular 0.9, 1.0, 1.1 or 1.3, such as 1.1%.
vi. G0-N0.2 to 1.2% of the population, such as 0.5 to 0.9%, in particular 0.7%;
vii. G2F 0.1 to 1% of the population, such as 0.3 to 0.7, in particular 0.5%;
and/or wherein at least 80% of the population is G0F.

52. A composition according to claim 35, wherein the population of glycans comprises afucosylated glycans (such as G0-N and/or G0) at 1.0 to 3.0% of the population, such as 1.5 to 2.6%, such as 1.8, 2.0 or 2.6%, in particular 2.6%.

53. A composition according to claim 35, wherein the population of glycans comprises galactosylated glycans (such as G1F, G1F′ and/or G2F), at 7 to 9%, such as 7.3, 7.7, 8.1 or 8.8%, in particular 7.7%.

54. A method of treating an inflammatory disorder, for example atopic dermatitis, such as moderate to severe atopic dermatitis, comprising administering a therapeutically effective amount of a composition according to claim 35 to a subject in need thereof.


SEQUENCE LISTING (64 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDRH3
  VARIANT 1: note = X denotes Phe, Met, Gln, Leu or Val
  VARIANT 6: note = X denotes Ser or Ala
  VARIANT 7: note = X denotes Phe, Leu, Ala or Met
  VARIANT 9: note = X denotes Tyr, Gln, Lys, Arg, Trp, His, Ala, Thr, Ser, Asn or Gly
XPNWGXXDX

SEQ ID NO: 4 - protein, 9 aa, synthetic construct
  REGION 1..9: note = CDR H3 SEQUENCE
FPNWGALDQ

SEQ ID NO: 5 - protein, 9 aa, synthetic construct
  REGION 1..9: note = CDR H3 SEQUENCE
VPNMGSLDT

SEQ ID NO: 6 - protein, 9 aa, synthetic construct
  REGION 1..9: note = CDR H3 SEQUENCE
FPNWGSMDA

SEQ ID NO: 7 - protein, 9 aa, synthetic construct
  REGION 1..9: note = CDR H3 SEQUENCE
FPNWGSLDH

SEQ ID NO: 8 - protein, 9 aa, synthetic construct
  REGION 1..9: note = CDR H3 SEQUENCE
MPNWGSFDY

SEQ ID NO: 9 - protein, 9 aa, synthetic construct
  REGION 1..9: note = CDR H3 SEQUENCE
MPNWGSFDT

SEQ ID NO: 10 - protein, 9 aa, synthetic construct
  REGION 1..9: note = CDR H3 SEQUENCE
MPNWGSLDH

SEQ ID NO: 11 - protein, 9 aa, synthetic construct
  REGION 1..9: note = CDR H3 SEQUENCE
MPNWGSFDS

SEQ ID NO: 12 - protein, 9 aa, synthetic construct
  REGION 1..9: note = CDR H3 SEQUENCE
MPNWGSLDT

SEQ ID NO: 13 - protein, 9 aa, synthetic construct
  REGION 1..9: note = CDR H3 SEQUENCE
MPNWGSLDA

SEQ ID NO: 14 - protein, 9 aa, synthetic construct
  REGION 1..9: note = CDR H3 SEQUENCE
MPNWGSLDN

SEQ ID NO: 15 - protein, 9 aa, synthetic construct
  REGION 1..9: note = CDR H3 SEQUENCE
MPNWGALDS

SEQ ID NO: 16 - protein, 9 aa, synthetic construct
  REGION 1..9: note = CDR H3 SEQUENCE
MPNWGSLDN

SEQ ID NO: 17 - protein, 9 aa, synthetic construct
  REGION 1..9: note = CDR H3 SEQUENCE
MPNWGSLDY

SEQ ID NO: 18 - protein, 9 aa, synthetic construct
  REGION 1..9: note = CDR H3 SEQUENCE
MPNWGSFDH

SEQ ID NO: 19 - protein, 9 aa, synthetic construct
  REGION 1..9: note = CDR H3 SEQUENCE
MPNWGSLDS

SEQ ID NO: 20 - protein, 9 aa, synthetic construct
  REGION 1..9: note = CDR H3 SEQUENCE
MPNWGSLDG

SEQ ID NO: 21 - protein, 9 aa, synthetic construct
  REGION 1..9: note = CDR H3 SEQUENCE
VPNWGSLDG

SEQ ID NO: 22 - protein, 28 aa, synthetic construct
  REGION 1..28: note = CDR H3 SEQUENCE
CARFPNWGSLDHWGQGTLVTVSSASIKG

SEQ ID NO: 23 - protein, 28 aa, synthetic construct
  REGION 1..28: note = CDR H3 SEQUENCE
CARMPNWGSLDHWGQGTLVTVSSASTKG

SEQ ID NO: 24 - protein, 28 aa, synthetic construct
  REGION 1..28: note = CDR H3 SEQUENCE
CARMPNWGSFDYWGQGTLVTVSSASIKG

SEQ ID NO: 25 - protein, 12 aa, synthetic construct
  REGION 1..12: note = CDR H3 SEQUENCE
VRMPNWGSLDHW

SEQ ID NO: 26 - protein, 27 aa, synthetic construct
  REGION 1..27: note = CDR H3 SEQUENCE
VRMPNWGSLDHWGQGTLVTVSSADIKG

SEQ ID NO: 27 - protein, 27 aa, synthetic construct
  REGION 1..27: note = CDR H3 SEQUENCE
ARMPNWGSLDHWGQGTLVTVSSASIKG

SEQ ID NO: 28 - protein, 25 aa, synthetic construct
  REGION 1..25: note = CDR H3 SEQUENCE
FPNWGSFDYWGQGTLVTVSSASIKG

SEQ ID NO: 29 - protein, 9 aa, synthetic construct
  REGION 1..9: note = CDR H3 SEQUENCE
VPNWGSLDA

SEQ ID NO: 30 - protein, 9 aa, synthetic construct
  REGION 1..9: note = CDR H3 SEQUENCE
FPNWGSFDY

SEQ ID NO: 31 - protein, 12 aa, synthetic construct
  REGION 1..12: note = CDRL1
RASQSISSSYLA

SEQ ID NO: 32 - protein, 7 aa, synthetic construct
  REGION 1..7: note = CDRL2
GASSRAT

SEQ ID NO: 33 - skipped

SEQ ID NO: 34 - protein, 5 aa, synthetic construct
  REGION 1..5: note = CDRL3 SEQUENCE
QRYAT

SEQ ID NO: 35 - protein, 5 aa, synthetic construct
  REGION 1..5: note = CDRL3 SEQUENCE
QRYST

SEQ ID NO: 36 - protein, 5 aa, synthetic construct
  REGION 1..5: note = CDRL3 SEQUENCE
QMYST

SEQ ID NO: 37 - protein, 5 aa, synthetic construct
  REGION 1..5: note = CDRL3 SEQUENCE
QQVGT

SEQ ID NO: 38 - protein, 5 aa, synthetic construct
  REGION 1..5: note = CDRL3 SEQUENCE
QQVST

SEQ ID NO: 39 - protein, 5 aa, synthetic construct
  REGION 1..5: note = CDRL3 SEQUENCE
QQYST

SEQ ID NO: 40 - protein, 5 aa, synthetic construct
  REGION 1..5: note = CDRL3 SEQUENCE
QSYST

SEQ ID NO: 41 - protein, 5 aa, synthetic construct
  REGION 1..5: note = CDRL3 SEQUENCE
QQYAT

SEQ ID NO: 42 - protein, 5 aa, synthetic construct
  REGION 1..5: note = CDRL3 SEQUENCE
QQYSS

SEQ ID NO: 43 - protein, 5 aa, synthetic construct
  REGION 1..5: note = CDRL3 SEQUENCE
QTYST

SEQ ID NO: 44 - protein, 5 aa, synthetic construct
  REGION 1..5: note = CDRL3 SEQUENCE
QQYGS

SEQ ID NO: 45 - protein, 5 aa, synthetic construct
  REGION 1..5: note = CDRL3 SEQUENCE
QQYAS

SEQ ID NO: 46 - protein, 5 aa, synthetic construct
  REGION 1..5: note = CDRL3 SEQUENCE
QQYEA

SEQ ID NO: 47 - protein, 5 aa, synthetic construct
  REGION 1..5: note = CDRL3 SEQUENCE
QQYET

SEQ ID NO: 48 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 61 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 9 aa, synthetic construct
  CARBOHYD 5: note = N-linked
EEQFNSTYR